(19) United States
(12) Patent Application Publication
(10) Pub. No.: US 2010/0132059 A1
(43) Pub. Date: May 27, 2010

(54) TEMPERATURE-INDUCED POLYNUCLEOTIDES AND USES THEREFOR

(75) Inventors: Kevin Michael MCCARTHY, Haverhill, MA (US); Robin Allison Heller-Harrison, Andover, MA (US); Mark William Leonard, Manchester, NH (US); Timothy Shea Charlebois, Andover, MA (US)

Correspondence Address: WYETH LLC;PATENT LAW GROUP, 5 GIRALDA FARMS, MADISON, NJ, 07940, US

(73) Assignee: Wyeth LLC, Madison, NJ (US)

(21) Appl. No.: 12/615,710

(22) Filed: Nov. 10, 2009

Related U.S. Application Data
(60) Provisional application No. 61/112,812, filed on Nov. 10, 2008.

(51) Int. Cl. A01K 67/00; C07K 2/00; C12N 5/10; C07H 21/00; C07H 21/04; C12N 15/74

(52) U.S. Cl. 800/13; 530/300; 536/23.1; 435/358; 536/24.5; 536/23.5; 536/24.1; 435/320.1

(57) ABSTRACT

The present invention is based on the discovery that the transcriptional activity of both hamster mammary tumor 7 (HMT-7) and hamster layilin genes are increased when Chinese Hamster Ovary (CHO) cells are cultured at temperatures lower than 37° C. The present invention provides novel genomic, cDNA, and protein sequences of HMT-7 and a novel genomic sequence for hamster layilin. Included in the genomic sequences are novel temperature-induced promoter sequences for each; two promoter sequences are provided for HMT-7 and one promoter sequence is provided for layilin. The invention additionally provides antisense and siRNA molecules to the nucleic acid molecules of HMT-7. The invention also provides genetically engineered expression vectors comprising the novel temperature-induced promoters of the invention, which are particularly useful as part of a mammalian temperature-inducible expression system. The invention also provides host cells and/or transgenic animals comprising the novel nucleic acid molecules of the invention. The invention further provides methods of using the polynucleotides of the invention, e.g., for temperature-induced transgene expression.

This application claims the benefit under 35 U.S.C. §119(E) to U.S. Provisional Application No. 61/112,812 filed Nov. 10, 2008 which is hereby incorporated by reference in its entirety.

### BACKGROUND

The present invention is directed toward temperature-induced polynucleotide sequences and uses thereof, e.g., as part of an inducible mammalian expression system.

Fundamental to the current study of biology is the ability to optimally culture and maintain cell lines. Of particular importance is the use of genetically engineered prokaryotic or eukaryotic cell lines to generate mass quantities of recombinant proteins. A recombinant protein may be used, e.g., in a biological study, or as a therapeutic compound for treating a particular ailment or disease.

The production of recombinant proteins for biopharmaceutical application typically requires vast numbers of cells and/or particular cell culture conditions that influence cell growth and/or expression. Production of recombinant proteins benefits from the use of an inducible expression system, i.e., a system that allows transgene expression to be induced under certain culture conditions (e.g., cell culture temperature, the presence of external agents (e.g., tetracycline, eckdysone, cumate, estrogen), etc.). Currently, there are few inducible mammalian expression systems, and the majority of the commercially available systems require the addition of an external agent; the only system that uses temperature to induce gene expression is restricted to use with bacterial cells (Qing et al. (2004) Nat. Biotechnol. 22:877-82; Carrao et al. (2003) Mol. Microbiol. 50:1349-60).

The present invention provides an inducible expression system that 1) may be used in mammalian cells and 2) allows the expression of transgene by a cell to be induced under a certain culture condition, in particular, when the cell is cultured at an inducing temperature.

### SUMMARY OF THE INVENTION

The present invention utilizes oligonucleotide microarray technology to identify genes and related sequences that are regulated in response to specific culture conditions, especially those conditions that result in optimal expression of transferred genes (transgenes), and consequently recombinant proteins, by genetically engineered cells or genetically engineered cell lines. In particular, the invention utilizes a hamster oligonucleotide array (see U.S. patent application Ser. Nos. 11/128,049 and 11/128,061) to identify genes that are induced under a specific culture temperature(s), e.g., genes expressed at a higher level by cells when the cells are cultured at temperatures below the physiological temperature of the cells (e.g., temperatures below 37° C., e.g., temperatures ranging from 25° C. to 34° C.).

One such gene of the invention is hamster mammary tumor-7, HMT-7. Thus, the invention provides an isolated polypeptide comprising an amino acid sequence selected from the group consisting of the amino acid sequence of HMT-7 (as set forth in SEQ ID NO:5) and the amino acid sequence of an active fragment of SEQ ID NO:5. The invention also provides an isolated nucleic acid molecule having a polynucleotide sequence that encodes the isolated polypeptide of HMT-7, e.g., wherein the nucleic acid molecule has a polynucleotide sequence selected from the group consisting of the polynucleotide sequence of HMT-7 cDNA (as set forth in SEQ ID NO:4), the polynucleotide sequence of the complement of SEQ ID NO:4, the polynucleotide sequence of a subsequence of SEQ ID NO:4, and the polynucleotide sequence of the complement of a subsequence of SEQ ID NO:4. In one embodiment of the invention, the isolated nucleic acid molecules having a polynucleotide sequence that encodes the isolated polypeptide of HMT-7 are operably linked to at least one expression control sequence, and may also be used to transform or transfect host cells of the invention and/or create nonhuman transgenic animals of the invention. The invention also provides an isolated nucleic acid molecule(s) that specifically hybridizes under highly stringent conditions to an isolated nucleic acid molecule(s) having a polynucleotide sequence that encodes the isolated polypeptide of HMT-7.

The invention also provides inhibitory polynucleotides that can alter the expression of HMT-7, e.g., an antisense oligonucleotide complementary to an mRNA corresponding to an isolated nucleic acid molecule having a polynucleotide sequence that encodes an isolated polypeptide of HMT-7, an siRNA molecule comprising at least one strand of RNA, wherein the one strand has a polynucleotide sequence complementary to an mRNA corresponding to an isolated nucleic acid molecule having a polynucleotide sequence that encodes the isolated polypeptide of HMT-7, etc.

The invention is also directed to an isolated gene that encodes HMT-7, e.g., an isolated allele of the HMT-7 gene, e.g., an isolated allele having a polynucleotide sequence selected from the group consisting of the polynucleotide sequence of SEQ ID NO:1, the polynucleotide sequence of the complement of SEQ ID NO:1, the polynucleotide sequence of a subsequence of SEQ ID NO:1, and the polynucleotide sequence of the complement of a subsequence of SEQ ID NO:1.

Another gene of the invention is hamster layilin. Thus, the invention also provides an isolated gene having a polynucleotide sequence that encodes hamster layilin, e.g., an isolated allele of the layilin gene, e.g., an isolated allele of the layilin gene having a polynucleotide sequence selected from the group consisting of the polynucleotide sequence of SEQ ID NO:6, the polynucleotide sequence of the complement of SEQ ID NO:6, the polynucleotide sequence of a subsequence of SEQ ID NO:6, and the polynucleotide sequence of the complement of a subsequence of SEQ ID NO:6.

In another embodiment, the invention is directed toward novel isolated polynucleotides encoding the promoters of HMT-7 or layilin. Thus, the invention provides an isolated temperature-induced promoter having a polynucleotide sequence selected from the group consisting of the polynucleotide sequence of an HMT-7 promoter and the polynucleotide sequence of a layilin promoter, e.g., an isolated temperature-induced promoter having a polynucleotide sequence selected from the group consisting of the polynucleotide sequence of an HMT-7 promoter (e.g., the polynucleotide sequence of P1-HMT-7 as set forth in SEQ ID NO:2, the polynucleotide sequence of P2-HMT-7 as set forth in SEQ ID NO:3, etc.), the polynucleotide sequence of a layilin promoter (e.g., the polynucleotide sequence of P-layilin as set forth in SEQ ID NO:7), the polynucleotide sequence of a small domain (of the HMT-7 gene) comprising P2-HMT-7 (e.g., the polynucleotide sequence as set forth in SEQ ID NO:16), and the polynucleotide sequence of a large domain (of the HMT-7 gene) comprising P2-HMT-7 (e.g., the polynucleotide sequence as set forth in SEQ ID NO: 17). In another embodiment, the invention provides a host cell transformed or transfected with an isolated temperature-induced promoter of the invention.

The invention also relates to the use of the temperature-induced promoters of the invention. Thus, in one embodiment, the isolated temperature-induced promoter regulates the expression of transgene in the transformed or transfected host cell. In another embodiment, the host cell is a CHO cell. The invention also provides temperature-inducible mammalian expression vectors comprising a temperature-induced promoter having a polynucleotide sequence selected from the group consisting of the polynucleotide sequence of an HMT-7 promoter and the polynucleotide sequence of a layilin promoter. In one embodiment, the temperature-induced promoter of a temperature-inducible mammalian expression vector of the invention has a polynucleotide sequence selected from the group consisting of the polynucleotide sequence of SEQ ID NO:2, the polynucleotide sequence of SEQ ID NO:3, the polynucleotide sequence of SEQ ID NO:7, the polynucleotide sequence of SEQ ID NO:16, and the polynucleotide sequence of SEQ ID NO:17. In another embodiment, the invention provides a host cell transformed or transfected with a temperature-inducible mammalian expression vector of the invention. In another embodiment, the host cell is a CHO cell.

The invention is also directed toward methods of using the temperature-induced mammalian expression vectors of the invention. In one embodiment, the invention provides a method of inducing transgene expression by a cell comprising the steps of introducing an expression vector into the cell, wherein the expression vector comprises a mammalian temperature-induced promoter, and wherein the temperature-induced promoter regulates the expression of the transgene; and culturing the cell at an inducing temperature. In one embodiment, the temperature-induced promoter has a polynucleotide sequence selected from the group consisting of the polynucleotide sequence of SEQ ID NO:2, the polynucleotide sequence of SEQ ID NO:3, the polynucleotide sequence of SEQ ID NO:7, the polynucleotide sequence of SEQ ID NO:16, and the polynucleotide sequence of SEQ ID NO:17. In one embodiment of the invention, the inducing temperature is below physiological temperature of the cell. In another embodiment of the invention, the inducing temperature is in a range of 25° C. to 34° C. In another embodiment, the invention also provides a kit comprising a mammalian expression vector, wherein the mammalian expression vector comprises a temperature-induced promoter having a polynucleotide sequence selected from the group consisting of the polynucleotide sequence of an HMT-7 promoter and the polynucleotide sequence of a layilin promoter.

One of skill in the art will recognize that the methods provided herein may be used to isolate other sequences differentially expressed under different culture conditions, e.g., promoters that may be useful in inducible expression system. Thus, in one embodiment, the invention provide a hamster sequence differentially expressed under different culture conditions, determined by a method comprising the steps of forming a first hybridization profile and a second hybridization profile, wherein the first hybridization profile is formed by incubating target nucleic acids prepared from a first cell with a first hamster oligonucleotide array, wherein the second hybridization profile is formed by incubating target nucleic acids prepared from a second cell with a second hamster oligonucleotide array identical to the first hamster oligonucleotide array, and wherein the first cell differs from the second cell with respect to culture condition; detecting the first and the second hybridization profiles; comparing the first and second hybridization profiles; and determining at least one hamster sequence with a differential expression level in the first hybridization profile relative to its expression level in the second hybridization level.

### BRIEF DESCRIPTION OF THE DRAWINGS

FIG. 1: Expression of HMT-7 or layilin by CHO cells cultured at temperatures between 37° C. and 28° C. as determined by real-time polymerase chain reaction. Shown in FIG. 1 is the amount (ng; y-axes) of (A) HMT-7 or (B) layilin RNA expressed by CHO cells cultured at 37° C. [symbol] , 34° C. [symbol] , 31° C. [symbol] or 28° C. [symbol] from four or eight experiments (Experiment; x-axes) as determined by real-time PCR.

FIG. 2: Diagram of the HMT-7 genomic locus. Shown in FIG. 2 is a cartoon schematic representation of the HMT-7 genomic locus that depicts the ATG start site for RIKEN 0610039N19 (N19 “start site”), the transcriptional start site for HMT-7 (Transcriptional start site), HMT-7 exons and corresponding peptide sequence (rectangles and black arrows, respectively), and the two predicted promoter sites (P1-HMT-7, P2-HMT-7; white arrows).

FIG. 3: Schematics of a control human placental alkaline phosphatase reporter construct and human placental alkaline phosphatase reporter constructs comprising domains comprising the P2-HMT-7 promoter. Cartoons are shown of the human placental alkaline phosphatase reporter constructs under the control of (FIG. 3A) no HMT-7 promoter, (FIG. 3B) a CMV promoter, (FIG. 3C) a small domain comprising P2-HMT-7, and (FIG. 3D) a large domain comprising P2-HMT-7.

FIG. 4: Expression of a human placental alkaline phosphatase reporter gene under the control of P2-HMT-7 by CHO cells cultured at temperatures between 37° C. and 31° C. as determined by real-time polymerase chain reaction. Shown in FIG. 4 is the amount of human placental alkaline phosphatase (SEAP) RNA over the amount of GAPDH RNA (ng of SEAP/ng of GAPDH; y-axis) expressed by CHO cells transfected with a reporter construct comprising SEAP under the control of a large domain comprising P2-HMT-7 (open columns), CHO cells transfected with a reporter construct comprising SEAP under the control of a small domain comprising P2-HMT-7(filled columns), or CHO cells transfected with a reporter construct comprising SEAP under the control of no promoter(cross-hatched columns) after culture at 37° C. or 31° C. (Temp; x-axis). At both temperatures, SEAP RNA was undetectable in pools of untransfected control CHO cells.

FIG. 5: Expression of a human placental alkaline phosphatase reporter gene under the control of a CMV promoter or a large domain comprising P2-HMT-7 by CHO cells cultured at 37° C. or 31° C. as determined by real-time polymerase chain reaction. Shown in FIG. 5 are relative expression levels of human placental alkaline phosphatase (SEAP) (ng of SEAP RNA/ng of GAPDH RNA; y-axis) from twenty-two clones of CHO cells (Clone number; x-axis) transfected with a reporter construct under the control of a large domain comprising P2-HMT-7 (▴,) or control CMV promoter (♦,▪) and cultured at 37° C. (♦,▴) or 31° C. (▪,).

### DETAILED DESCRIPTION OF THE INVENTION

The inventors used a hamster oligonucleotide array (see U.S. patent application Ser. Nos. 11/128,049 and 11/128,061, both of which are hereby incorporated by reference herein in their entirety) to identify genes that are induced under a specific culture condition(s) and the temperature-induced promoters of these genes, which may thus may be used as part of an inducible mammalian expression system to regulate transgene expression by a cell in such a way that such expression may be induced, e.g., increased, in response to a particular cell culture condition, e.g., temperature. Genes that were differentially expressed (e.g., had a two-fold greater expression level) at a temperature below physiological temperature were identified (Example 1). Real-time PCR and Northern Blot analysis confirmed the differential expression of the gene sequences at the different culture conditions (Example 2). Further characterization of the genes (Example 3) identified putative promoter sequences that were used to create a temperature-inducible mammalian expression vector (Example 4), which may be used to induce recombinant gene expression at an inducing temperature (Example 5). Accordingly, the present invention provides the polynucleotide sequences (and subsequences) of genes that are induced, e.g., expressed at higher levels, by cells cultured at temperatures below the physiological temperature of the cell. The present invention also provides the polynucleotide sequences of subsequences of the gene sequence (e.g., promoter sequences and/or enhancer sequences for the genes) that may be used to regulate the expression of a transgene. In particular, these temperature-induced promoters may be used to induce higher expression by a cell of a transgene (which is under the control of such a temperature-induced promoter) when the cell is cultured at an inducing temperature, e.g., a temperature below the physiological temperature of the cell. Additionally, the present invention provides temperature-inducible expression vectors that comprise the temperature-induced promoters of the invention, and methods of using such expression vectors.

### Isolated Polynucleotides and Polypeptides

Thus, the invention provides purified and isolated polynucleotide sequences of two genes that are induced, e.g., have higher expression levels, in CHO cells cultured at inducing temperatures, e.g., temperatures below the physiological temperature of CHO cells, compared to the expression levels of the two genes by CHO cells cultured at a physiological temperature, e.g., 37° C. These genes provide regulatory sequences (e.g., coding regions, promoters, enhancers, termination signals, etc.) that are preferably suitable targets for regulating expression of, e.g., a transgene, by a cell. The genes, polynucleotides, proteins, and polypeptides of the present invention include, but are not limited to, the gene sequences of hamster mammary tumor-7 (HMT-7) and its homologs, and hamster layilin and its homologs.

Accordingly, the present invention provides novel isolated and purified polynucleotides that are either or both 1) differentially expressed by cells depending on the cell culture temperature, and thus, 2) may be used as part of an inducible mammalian vector expression system for the regulation of a cell phenotype, e.g., transgene expression. It is also part of the invention to provide inhibitory polynucleotides to the novel isolated and purified polynucleotides of the invention, which may be used, e.g., as antagonists to the novel isolated and purified polynucleotides of the invention.

Nucleic acids according to the present invention may comprise DNA or RNA and may be wholly or partially synthetic. Reference to nucleotide sequences as set out herein encompass DNA molecules with the specified sequences or genomic equivalents (e.g., complementary sequences), as well as RNA molecules corresponding to the specified sequences in which T is substituted with U, unless context requires otherwise.

For example, the invention provides novel purified and isolated polynucleotides encoding hamster mammary tumor-7 (HMT-7), HMT-7 promoters and/or HMT-7 enhancers, etc. Preferred DNA sequences of the invention include genomic, cDNA and chemically synthesized DNA sequences.

The nucleotide sequence(s) of a novel gene, e.g., genomic DNA, encoding hamster mammary tumor-7, designated HMT-7 genomic DNA, has and/or consists essentially of the nucleotide sequence set forth in SEQ ID NO:1. Polynucleotides of the present invention also include polynucleotides that hybridize under stringent conditions to SEQ ID NO:1, or its complement, and/or encode polypeptides that retain substantial biological activity (i.e., active fragments) of full-length HMT-7. Polynucleotides of the present invention also include continuous portions of the sequence set forth in SEQ ID NO:1 comprising at least 21 consecutive nucleotides.

The nucleotide sequences of two novel HMT-7 promoters, designated P1-HMT-7 and P2-HMT-7, have and/or consist essentially of the nucleotide sequences set forth in SEQ ID NO:2 and SEQ ID NO:3, respectively. SEQ ID NO:2 is the nucleotide sequence of nucleotides 5616-5762 of SEQ ID NO:1, and SEQ ID NO:3 is the nucleotide sequence of nucleotides 2423-2673 of SEQ ID NO:1. Polynucleotides of the present invention also include polynucleotides that hybridize under stringent conditions to SEQ ID NOs:2 or 3, and/or complements thereof, and/or those that retain substantial biological activity of P1-HMT-7 or P2-HMT-7. Polynucleotides of the present invention also include continuous portions of the sequence set forth in SEQ ID NO:2 or SEQ ID NO:3 comprising at least 21 consecutive nucleotides.

The nucleotide sequence(s) of a novel cDNA encoding HMT-7, designated HMT-7 cDNA, has and/or consists essentially of the nucleotide sequence set forth in SEQ ID NO:4. Polynucleotides of the present invention also include polynucleotides that hybridize under stringent conditions to SEQ ID NO:4, or its complement, and/or encode polypeptides that retain substantial biological activity of full-length HMT-7. Polynucleotides of the present invention also include continuous portions of the sequence set forth in SEQ ID NO:4 comprising at least 21 consecutive nucleotides.

The amino acid sequence(s) of the novel HMT-7 protein is set forth in SEQ ID NO:5. Polypeptides of the present invention also include continuous portions of the sequence set forth in SEQ ID NO:5 comprising at least seven consecutive amino acids. A preferred polypeptide of the present invention includes any continuous portion of the sequence set forth in SEQ ID NO:5 that retains substantial biological activity of full-length HMT-7, i.e., an active fragment of HMT-7. Polynucleotides of the present invention also include, in addition to those polynucleotides of hamster origin described above, polynucleotides that encode the amino acid sequence set forth in SEQ ID NO:5 or a continuous portion thereof, and that differ from the polynucleotides described above only due to the well-known degeneracy of the genetic code.

In another embodiment, the invention provides the novel purified and isolated polynucleotides encoding hamster layilin, layilin promoters and/or layilin enhancers, etc. Preferred DNA sequences of the invention include genomic, cDNA and chemically synthesized DNA sequences.

The nucleotide sequence(s) of a novel gene, i.e., genomic DNA, encoding hamster layilin, designated layilin genomic DNA, has and/or consists essentially of the nucleotide sequence set forth in SEQ ID NO:6. Polynucleotides of the present invention also include polynucleotides that hybridize under stringent conditions to SEQ ID NO:6, or its complement, and/or encode polypeptides that retain substantial biological activity (i.e., active fragments) of full-length layilin. Polynucleotides of the present invention also include continuous portions of the sequence set forth in SEQ ID NO:6 comprising at least 21 consecutive nucleotides.

The nucleotide sequence(s) of a novel layilin promoter, designated P-layilin, has and/or consists essentially of the nucleotide sequence set forth in SEQ ID NO:7. Polynucleotides of the present invention also include polynucleotides that hybridize under stringent conditions to SEQ ID NO:7, complements thereof, and/or retain substantial biological activity of P-layilin. Polynucleotides of the present invention also include continuous portions of the sequence set forth in SEQ ID NO:7 comprising at least 21 consecutive nucleotides.

The isolated polynucleotides of the present invention may be used as hybridization probes and primers to identify and isolate nucleic acids having sequences identical to or similar to those encoding the disclosed polynucleotides. Hybridization methods for identifying and isolating nucleic acids include polymerase chain reaction (PCR), Southern hybridizations, in situ hybridization and Northern hybridization, and are well known to those skilled in the art.

Hybridization reactions can be performed under conditions of different stringencies. The stringency of a hybridization reaction includes the difficulty with which any two nucleic acid molecules will hybridize to one another. Preferably, each hybridizing polynucleotide hybridizes to its corresponding polynucleotide under reduced stringency conditions, more preferably stringent conditions, and most preferably highly stringent conditions. Examples of stringency conditions are shown in Table 1 below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE 1

| | Poly- | | Hybridization | |
|---|---|---|---|---|
| Stringency | nucleotide | Hybrid | Temperature and | Wash Temperature |
| Condition | Hybrid | Length (bp)¹ | Buffer² | and Buffer² |
| A | DNA:DNA | >50 | 65° C.; 1X SSC -or- | 65° C.; 0.3X SSC |
| | | | 42° C.; 1X SSC, 50% | |
| | | | formamide | |
| B | DNA:DNA | <50 | T_{B}*; 1X SSC | T_{B}*; 1X SSC |
| C | DNA:RNA | >50 | 67° C.; 1X SSC -or- | 67° C.; 0.3X SSC |
| | | | 45° C.; 1X SSC, 50% | |
| | | | formamide | |
| D | DNA:RNA | <50 | T_{D}*; 1 × SSC | T_{D}*; 1X SSC |
| E | RNA:RNA | >50 | 70° C.; 1X SSC | 70° C.; O.3xSSC |
| | | | -or- | |
| | | | 50° C.; 1X SSC, 50% | |
| | | | formamide | |
| F | RNA:RNA | <50 | T_{F}*; 1X SSC | T_{f}*; X SSC |
| G | DNA:DNA | >50 | 65° C.; 4X SSC | 65° C.; 1X SSC |
| | | | -or- | |
| | | | 42° C.; 4X SSC, 50% | |
| | | | formamide | |
| H | DNA:DNA | <50 | T_{H}*; 4X SSC | T_{H}*; 4X SSC |
| I | DNA:RNA | >50 | 67° C.; 4X SSC | 67° C.; 1X SSC |
| | | | -or- | |
| | | | 45° C.; 4X SSC, 50% | |
| | | | formamide | |
| J | DNA:RNA | <50 | T_{J}*; 4X SSC | T_{J}*; 4X SSC |
| K | RNA:RNA | >50 | 70° C.; 4X SSC | 67° C.; 1X SSC |
| | | | -or- | |
| | | | 50° C.; 4X SSC, 50% | |
| | | | formamide | |
| L | RNA:RNA | <50 | T_{L}*; 2X SSC | T_{L}*; 2X SSC |
| M | DNA:DNA | >50 | 50° C.; 4X SSC | 50° C.; 2X SSC |
| | | | -or- | |
| | | | 40° C.; 6X SSC, 50% | |
| | | | formamide | |
| N | DNA:DNA | <50 | T_{N}*; 6X SSC | T_{N}*; 6X SSC |
| O | DNA:RNA | >50 | 55° C.; 4X SSC | 55° C.; 2X SSC |
| | | | -or- | |
| | | | 42° C.; 6X SSC, 50% | |
| | | | formamide | |
| P | DNA:RNA | <50 | T_{P}*; 6X SSC | T_{P}*; 6X SSC |
| Q | RNA:RNA | >50 | 60° C.; 4X SSC -or- | 60° C.; 2X SSC |
| | | | 45° C.; 6X SSC, 50% | |
| | | | formamide | |
| R | RNA:RNA | <50 | T_{R}*; 4X SSC | T_{R}*; 4X SSC |

¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity.
²SSPE (1xSSPE is 0.15M NaCl, 10 mM NaH₂PO₄, and 1.25 mM EDTA, pH 7.4) can be substituted for SSC (1xSSC is 0.15M NaCl and 15 mM sodium citrate) in the hybridization and wash buffers; washes are performed for 15 minutes after hybridization is complete.
T_{B}*-T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between 18 and 49 base pairs in length, Tₘ(° C.) = 81.5 + 16.6(log₁₀Na⁺) + 0.41(% G + C) − (600/N), where N is the number of bases in the hybrid, and Na⁺ is the concentration of sodium ions in the hybridization buffer (Na⁺ for 1xSSC = 0.165M).
Additional examples of stringency conditions for polynucleotide hybridization are provided in Sambrook et al. (1989) Molecular Cloning: A Laboratory Manual, Chs. 9 & 11, Cold Spring Harbor Laboratory Press, Cold Spring Harbor, NY, and Ausubel et al., Eds. (1995) Current Protocols in Molecular Biology, Sects. 2.10 & 6.3-6.4, John Wiley & Sons, Inc., herein incorporated by reference.

Generally, and as stated above, the isolated polynucleotides of the present invention may also be used as hybridization probes and primers to identify and isolate DNAs homologous to the disclosed polynucleotides. These homologs are polynucleotides isolated from different species than those of the disclosed polynucleotides, or within the same species, but with significant sequence similarity to the disclosed polynucleotides. Preferably, polynucleotide homologs have at least 60% sequence identity (more preferably, at least 75% identity; most preferably, at least 90% identity) with the disclosed polynucleotides. Preferably, homologs of the disclosed polynucleotides are those isolated from mammalian species.

The isolated polynucleotides of the present invention may also be used as hybridization probes and primers to identify cells and tissues that express the polynucleotides of the present invention and the conditions under which they are expressed.

Additionally, the polynucleotides of the present invention may be used to alter (e.g., enhance, reduce, or modify) the expression of the genes corresponding to HMT-7 polynucleotide sequences of the present invention in a cell or organism. These corresponding genes are the genomic DNA sequences of the present invention that are transcribed to produce the mRNAs from which the HMT-7 polynucleotide sequences of the present invention are derived.

### Inhibitory Polynucleotides

Altered expression of the HMT-7 or layilin polynucleotide sequences encompassed by the present invention in a cell or organism may be achieved through the use of various inhibitory polynucleotides, such as antisense polynucleotides, ribozymes that bind and/or cleave the mRNA transcribed from the genes of the invention, triplex-forming oligonucleotides that target regulatory regions of the genes, and short interfering RNA that causes sequence-specific degradation of target mRNA (e.g., Galderisi et al. (1999) J. Cell. Physiol. 181:251-57; Sioud (2001) Curr. Mol. Med. 1:575-88; Knauert and Glazer (2001) Hum. Mol. Genet. 10:2243-51; Bass (2001) Nature 411:428-29). It should be noted that, although the use of inhibitory polynucleotides have been described for genes homologous to layilin (see, e.g., U.S. Published Patent Application No. 2005/0136435 and International Published Patent Application No. WO 2005/060996 A2), the inventors do not know of any published reports of inhibitory polynucleotides to layilin.

The inhibitory antisense or ribozyme polynucleotides of the invention can be complementary to an entire coding strand of a gene of the invention, or to only a portion thereof. Alternatively, inhibitory polynucleotides can be complementary to a noncoding region of the coding strand of a gene of the invention. The inhibitory polynucleotides of the invention can be constructed using chemical synthesis and/or enzymatic ligation reactions using procedures well known in the art. The nucleoside linkages of chemically synthesized polynucleotides can be modified to enhance their ability to resist nuclease-mediated degradation, as well as to increase their sequence specificity. Such linkage modifications include, but are not limited to, phosphorothioate, methylphosphonate, phosphoroamidate, boranophosphate, morpholino, and peptide nucleic acid (PNA) linkages (Galderisi et al., supra; Heasman (2002) Dev. Biol. 243:209-14; Mickelfield (2001) Curr. Med. Chem. 8:1157-79). Alternatively, antisense molecules can be produced biologically using an expression vector into which a polynucleotide of the present invention has been subcloned in an antisense (i.e., reverse) orientation.

In yet another embodiment, the antisense polynucleotide molecule of the invention is an α-anomeric polynucleotide molecule. An α-anomeric polynucleotide molecule forms specific double-stranded hybrids with complementary RNA in which, contrary to the usual β-units, the strands run parallel to each other. The antisense polynucleotide molecule can also comprise a 2′-o-methylribonucleotide or a chimeric RNA-DNA analogue, according to techniques that are known in the art.

The inhibitory triplex-forming oligonucleotides (TFOs) encompassed by the present invention bind in the major groove of duplex DNA with high specificity and affinity (Knauert and Glazer, supra). Expression of the genes of the present invention can be inhibited by targeting TFOs complementary to the regulatory regions of the genes (i.e., the promoter and/or enhancer sequences) to form triple helical structures that prevent transcription of the genes.

In one embodiment of the invention, the inhibitory polynucleotides of the present invention are short interfering RNA (siRNA) molecules. These siRNA molecules are short (preferably 19-25 nucleotides; most preferably 19 or 21 nucleotides), double-stranded RNA molecules that cause sequence-specific degradation of target mRNA. This degradation is known as RNA interference (RNAi) (e.g., Bass (2001) Nature 411:428-29). Originally identified in lower organisms, RNAi has been effectively applied to mammalian cells and has recently been shown to prevent fulminant hepatitis in mice treated with siRNA molecules targeted to Fas mRNA (Song et al. (2003) Nat. Med. 9:347-51). In addition, intrathecally delivered siRNA has recently been reported to block pain responses in two models (agonist-induced pain model and neuropathic pain model) in the rat (Dorn et al. (2004) Nucleic Acids Res. 32(5):e49).

The siRNA molecules of the present invention can be generated by annealing two complementary single-stranded RNA molecules together (one of which matches a portion of the target mRNA) (Fire et al., U.S. Pat. No. 6,506,559) or through the use of a single hairpin RNA molecule that folds back on itself to produce the requisite double-stranded portion (Yu et al. (2002) Proc. Natl. Acad. Sci. USA 99:6047-52). The siRNA molecules can be chemically synthesized (Elbashir et al. (2001) Nature 411:494-98) or produced by in vitro transcription using single-stranded DNA templates (Yu et al., supra). Alternatively, the siRNA molecules can be produced biologically, either transiently (Yu et al., supra; Sui et al. (2002) Proc. Natl. Acad. Sci. USA 99:5515-20) or stably (Paddison et al. (2002) Proc. Natl. Acad. Sci. USA 99:1443-48), using an expression vector(s) containing the sense and antisense siRNA sequences. Recently, reduction of levels of target mRNA in primary human cells, in an efficient and sequence-specific manner, was demonstrated using adenoviral vectors that express hairpin RNAs, which are further processed into siRNAs (Arts et al. (2003) Genome Res. 13:2325-32).

The siRNA molecules targeted to the polynucleotides of the present invention can be designed based on criteria well known in the art (e.g., Elbashir et al. (2001) EMBO J. 20:6877-88). For example, the target segment of the target mRNA should begin with AA (preferred), TA, GA, or CA; the GC ratio of the siRNA molecule should be 45-55%; the siRNA molecule should not contain three of the same nucleotides in a row; the siRNA molecule should not contain seven mixed G/Cs in a row; and the target segment should be in the ORF region of the target mRNA and should be at least 75 by after the initiation ATG and at least 75 by before the stop codon. siRNA molecules targeted to the polynucleotides of the present invention can be designed by one of ordinary skill in the art using the aforementioned criteria or other known criteria.

Altered expression of the polynucleotide sequences of the present invention in a cell or organism may also be achieved through the creation of nonhuman transgenic animals into whose genomes polynucleotides of the present invention have been introduced. Such transgenic animals include animals that have multiple copies of a gene (i.e., the transgene) of the present invention. A tissue-specific regulatory sequence(s) may be operably linked to a polynucleotide of present invention to direct its expression to particular cells or a particular developmental stage. In another embodiment, transgenic nonhuman animals can be produced that contain selected systems that allow for regulated expression of the transgene. One example of such a system known in the art is the cre/loxP recombinase system of bacteriophage P1. Methods for generating transgenic animals via embryo manipulation and microinjection, particularly animals such as mice, have become conventional and are well known in the art (e.g., Bockamp et al. (2002) Physiol. Genomics 11:115-32). In at least one embodiment of the invention, the nonhuman transgenic animal comprises at least one HMT-7 polynucleotide sequence.

Altered expression of the genes of the present invention in a cell or organism may also be achieved through the creation of animals whose endogenous genes corresponding to the polynucleotides of the present invention have been disrupted through insertion of extraneous polynucleotides sequences (i.e., a knockout animal). The coding region of the endogenous gene may be disrupted, thereby generating a nonfunctional protein. Alternatively, the upstream regulatory region of the endogenous gene may be disrupted or replaced with different regulatory elements, resulting in the altered expression of the still-functional protein. Methods for generating knockout animals include homologous recombination and are well known in the art (e.g., Wolfer et al. (2002) Trends Neurosci. 25:336-40).

The isolated polynucleotides of the present invention may be operably linked to an expression control sequence such as the pMT2 and pED expression vectors for recombinant production of the polypeptides encoded by the polynucleotides of the invention. General methods of expressing recombinant proteins are well known in the art.

A number of cell types may act as suitable host cells for recombinant expression of the polypeptides encoded by the polynucleotides of the invention. Mammalian host cells include, but are not limited to, e.g., COS cells, CHO cells, 293 cells, A431 cells, 3T3 cells, CV-1 cells, HeLa cells, L cells, BHK21 cells, HL-60 cells, U937 cells, HaK cells, Jurkat cells, normal diploid cells, cell strains derived from in vitro culture of primary tissue, and primary explants.

Alternatively, it may be possible to recombinantly produce the polypeptides encoded by polynucleotides of the present invention in lower eukaryotes such as yeast or in prokaryotes. Potentially suitable yeast strains include Saccharomyces cerevisiae, Schizosaccharomyces pombe, Kluyveromyces strains, and Candida strains. Potentially suitable bacterial strains include Escherichia coli, Bacillus subtilis, and Salmonella typhimurium. If the polypeptides are made in yeast or bacteria, it may be necessary to modify them by, e.g., phosphorylation or glycosylation of appropriate sites, in order to obtain functionality. Such covalent attachments may be accomplished using well-known chemical or enzymatic methods.

The polypeptides encoded by polynucleotides of the present invention may also be recombinantly produced by operably linking the isolated polynucleotides of the present invention to suitable control sequences in one or more insect expression vectors, such as baculovirus vectors, and employing an insect cell expression system. Materials and methods for baculovirus/Sf9 expression systems are commercially available in kit form (e.g., the MaxBac® kit, Invitrogen, Carlsbad, Calif.).

Following recombinant expression in the appropriate host cells, the polypeptides encoded by polynucleotides of the present invention may then be purified from culture medium or cell extracts using known purification processes, such as gel filtration and ion exchange chromatography. Purification may also include affinity chromatography with agents known to bind the polypeptides encoded by the polynucleotides of the present invention. These purification processes may also be used to purify the polypeptides from natural sources.

Alternatively, the polypeptides encoded by polynucleotides of the present invention may also be recombinantly expressed in a form that facilitates purification. For example, the polypeptides may be expressed as fusions with proteins such as maltose-binding protein (MBP), glutathione-S-transferase (GST), or thioredoxin (TRX). Kits for expression and purification of such fusion proteins are commercially available from New England BioLabs (Beverly, Mass.), Pharmacia (Piscataway, N.J.), and Invitrogen (Carlsbad, Calif.), respectively. The polypeptides encoded by polynucleotides of the present invention can also be tagged with a small epitope and subsequently identified or purified using a specific antibody to the epitope. A preferred epitope is the FLAG epitope, which is commercially available from Eastman Kodak (New Haven, Conn.).

The polypeptides encoded by polynucleotides of the present invention may also be produced by known conventional chemical synthesis. Methods for chemically synthesizing the polypeptides encoded by polynucleotides of the present invention are well known to those skilled in the art. Such chemically synthetic polypeptides may possess biological properties in common with the natural, purified polypeptides, and thus may be employed as biologically active or immunological substitutes for the natural polypeptides.

### Temperature-Induced Promotors and Mammalian Expression Vectors

In addition to providing novel cDNA and amino acid sequences for HMT-7, the inventors also provide a novel sequence for the gene encoding HMT-7 (i.e., DNA having a polynucleotide sequence that encodes the HMT-7 polypeptide chain, and including regions preceding and following the coding DNA (e.g., promoters, enhancers, UTRs, etc.) as well as introns between the exons). The inventors also provide a novel sequence for a gene encoding hamster layilin (i.e., DNA having a polynucleotide sequence that encodes the layilin polypeptide chain; and including regions preceding and following the coding DNA (e.g., promoters, enhancers, UTRs, etc.) as well as introns between the exons); the cDNA and amino acid sequences of hamster layilin may be found in the GenBank database with accession numbers AF09673 and AAC68695, respectively. In providing these novel gene sequences, the inventors also provide putative promoter sequences for both HMT-7 and layilin. As CHO cells express HMT-7 or layilin at higher levels when the cells are cultured at temperatures below physiological temperature, it is expected that promoters of HMT-7 and layilin may be used as temperature-induced promoters. In fact, the inventors demonstrate that an HMT-7 promoter (e.g., an HMT-7 promoter having and/or consisting essentially of the nucleotide sequence set forth in SEQ ID NO:3) may be used to induce higher expression of a transgene under its control by a cell when the cell is cultured below its physiological temperature (Example 5). Additionally, it is believed that a layilin promoter (e.g., a layilin promoter having and/or consisting essentially of the nucleotide sequence set forth in SEQ ID NO:7) may be similarly used (Example 6). Consequently, the invention provides temperature-induced promoters.

As discussed above, the nucleotide sequence of an HMT-7 promoter may have and/or consist essentially of the nucleotide sequence of SEQ ID NO:2 or SEQ ID NO:3; the nucleotide sequence of a layilin promoter may have and/or consist essentially of the nucleotide sequence of SEQ ID NO:7. These promoters were determined using computer algorithms that predict promoter regions, e.g., based on well-known characteristics of the promoter sequences, such as homology to other known promoter sequences. Using these characteristics, a skilled artisan will be able to recognize and identify other promoters for HMT-7 or layilin, which may or may not be found in SEQ ID NO:1 and SEQ ID NO:6, respectively. Additionally, using well-known methods including the methods provided herein (e.g., those employing reporter assays and culturing of cells under different temperatures), such a skilled artisan will also be able to determine whether the identified promoters are cold-induced promoters and/or the efficacy of such temperature-induced promoters. Such temperature-induced promoters are considered within the scope of the invention.

Furthermore, a skilled artisan will be able to use well-known recombinant DNA techniques to recombine a temperature-induced promoter of the invention with a transgene such that the temperature-induced promoter will act as a regulatory sequence to the transgene, i.e., such that the temperature-induced promoter will induce transcription of the transgene (and perhaps ultimately expression of a recombinant protein) at temperatures that also induce the temperature-induced promoter. One of skill in the art will recognize that such a recombined temperature-induced promoter-transgene construct may be introduced into a host cell alone, or more easily as part of a recombinant expression vector. Additionally, a skilled artisan will recognize that a transgene is not limited to the reporter gene used herein, or to reporter genes in general, i.e., that most genes and/or cDNAs encoding a polypeptide may be placed under the regulation of a temperature-induced promoter of the invention.

A skilled artisan will recognize that the term “expression vector,” as used herein, is intended to refer to a nucleic acid molecule capable of transporting another nucleic acid to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., nonepisomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”).

The term “regulatory sequence” is intended to encompass the temperature-induced promoters of the invention, other promoters, enhancers and other expression control elements (e.g., polyadenylation signals) that control the transcription or translation of a transgene. Such regulatory sequences are described, for example, in Goeddel, Gene Expression Technology: Methods in Enzymology 185, Academic Press, San Diego, Calif. (1990). It will be appreciated by those skilled in the art that the design of an expression vector of the invention, including the selection of other regulatory sequences in addition to the temperature-induced promoters of the invention, may depend on such factors as the choice of the host cell to be transformed, the level of expression of protein desired, etc. Other regulatory sequences that may be included in a recombinant expression vector of the invention (i.e., an expression vector comprising a temperature-induced promoter of the invention) for mammalian host cell expression are preferably viral elements that direct high levels of protein expression in mammalian cells, such as promoters and/or enhancers derived from FF-1a promoter and BGH poly A, cytomegalovirus (CMV) (such as the CMV promoter/enhancer), Simian Virus 40 (SV40) (such as the SV40 promoter/enhancer), adenovirus (e.g., the adenovirus major late promoter (AdMLP)), and polyoma. For further description of viral regulatory elements, and sequences thereof, see, e.g., U.S. Pat. No. 5,168,062 by Stinski, U.S. Pat. No. 4,510,245 by Bell et al., and U.S. Pat. No. 4,968,615 by Schaffner et al.

The recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr⁻ host cells with methotrexate selection/amplification) and the neo gene (for G418 selection).

### Methods of Using Temperature-Inducible Mammalian Expression Vectors

The invention also provides methods of using a temperature-induced promoter of the invention (e.g., as part of an expression vector of the invention) to regulate the expression of a transgene, e.g., to induce the expression of the transgene by a cell by culturing the cell at an inducing temperature. For example, the invention provides a method of inducing transgene expression by a cell comprising the steps of (1) introducing an expression vector into the cell, wherein the expression vector comprises a mammalian temperature-induced promoter (e.g., a temperature-induced promoter having and/or consisting essentially of a polynucleotide sequence selected from the group consisting of the polynucleotide sequence of an HMT-7 promoter and the polynucleotide sequence of a layilin promoter), and wherein the temperature-induced promoter regulates the expression of the transgene; and (2) culturing the cell at an inducing temperature. In one embodiment of the invention, the polynucleotide sequence of the HMT-7 promoter has and/or consists essentially of the polynucleotide sequence of SEQ ID NO:2. In another embodiment of the invention, the polynucleotide sequence of the HMT-7 promoter has and/or consists essentially of the polynucleotide sequence of SEQ ID NO:3. In a further embodiment of the invention, the polynucleotide sequence of the layilin promoter has and/or consists essentially of the polynucleotide sequence of SEQ ID NO:7.

Any available technique for the introduction of a temperature-induced promoter of the invention (or expression vector(s) comprising a temperature-induced promoter of the invention) into host cells or organisms will be well known by one of ordinary skill in the art and may be used. For example, if synthesized chemically or by in vitro enzymatic synthesis, the temperature-induced promoter and/or expression vector of the invention may be purified prior to introduction into a host cell or organism. For example, the temperature-induced promoter and/or expression vector may be purified from a mixture by extraction with a solvent or resin, precipitation, electrophoresis, chromatography, or a combination thereof. Alternatively, the temperature-induced promoter and/or expression vector may be used with no purification, or with a minimum of purification, to avoid losses due to sample processing. The temperature-induced promoter and/or expression vector may be dried for storage or dissolved in an aqueous solution. The solution may contain buffers or salts to promote annealing and/or stabilization of the temperature-induced promoter and/or expression vector. If purified, the temperature-induced promoter and/or expression vector may be directly introduced into the cell, introduced extracellularly into a cavity or interstitial space or into the circulation of an organism, introduced orally, or introduced by bathing a cell or organism in a solution comprising a temperature-induced promoter and/or expression vector of the invention. Physical methods of introducing nucleic acids include injection of a solution comprising a temperature-induced promoter and/or expression vector of the invention, bombardment by particles covered by a temperature-induced promoter and/or expression vector of the invention, soaking or bathing the cell or organism in the solution, or electroporation.

For eukaryotic cells, suitable techniques for the introduction of a temperature-induced promoter(s) and/or expression vector(s) that comprises a temperature-induced promoter of the invention may include calcium phosphate transfection, DEAE Dextran, electroporation, liposome-mediated transfection, and transduction using retrovirus or other viruses, e.g., vaccinia. In a preferred embodiment, a viral construct packaged into a viral particle accomplishes both efficient introduction of a temperature-induced promoter and/or expression vector of the invention into the cell and transcription of a transgene regulated by a temperature-induced promoter. Additionally, the temperature-induced promoter and/or expression vector of the invention may be introduced along with components that perform one or more of the following activities: enhance uptake by the cell, promote stability of the temperature-induced promoter and/or expression vector, etc. Finally, the introduction may be followed by causing or allowing expression from the temperature-induced promoter, e.g., by culturing host cells at an inducing temperature. In one embodiment of the invention, the inducing temperature is below the physiological temperature of the host cell. In another embodiment of the invention, the inducing temperature is approximately 31° C.

Induction of expression refers to an observable increase in the level of transgene products (e.g., mRNA and/or protein), and may be detected by examination of the outward properties of the host cell or organism, or by biochemical techniques such as hybridization reactions (e.g., Northern blot analysis, RNase protection assays, microarray analysis, etc.), reverse transcription and polymerase chain reactions, binding reactions (e.g., Western blots, ELISA, FACS, etc.), reporter assays, drug resistance assays, etc. Depending on the method of detection, regulation of a transgene by a temperature-induced promoter and/or expression vector of the invention should induce a greater than 5%, 10%, 33%, 50%, 90%, 95% or 99% increase in the expression of the transgene by a host cell cultured at an inducing temperature (e.g., a temperature below the physiological temperature of the cell) compared to the expression of the transgene by the host cell cultured at the physiological temperature of the host cell. Additionally, treatment of a population of host cells according to a method provided herein may result in a fraction of the cells (e.g., at least 2%, 5%, 10%, 20%, 50%, 75%, 90%, 95%, or 99% of treated cells) exhibiting induced expression of a transgene regulated by a temperature-induced promoter and/or expression vector of the invention. Increasing the dose of the temperature-induced promoter and/or expression vector of the invention may increase the amount of induction detected. A skilled artisan will recognize that quantification of expression of the transgene in treated cell(s) or organism(s) may show dissimilar levels of induction at the mRNA level compared to the protein level. As an example, although the efficiency of inhibition may be determined by detecting the mRNA level of the gene of interest, e.g., by Northern blot analysis, a preferred method of determining the level of inhibition is by detecting the level of protein.

The temperature-induced promoters and/or expression vectors of the invention may be introduced into a host cell or organism, as described above, in sufficient amounts to allow introduction of at least one copy of a temperature-induced promoter into the cell. Higher doses (e.g., at least 5, 10, 100, 500, or 1000 copies per cell) of a temperature-induced promoter and/or expression vector of the invention may yield more effective induction at the inducing temperature.

The entire contents of all references, patents, patent applications, and publications cited in this application are hereby incorporated by reference herein.

### Examples

The Examples which follow are set forth to aid in the understanding of the invention but are not intended to, and should not be construed to, limit its scope in any way. The Examples do not include detailed descriptions of conventional methods, such as, real-time polymerase chain reaction (PCR), cell culture, RNA quantification or those methods employed in the construction of vectors, the insertion of genes encoding the polypeptides into such vectors and plasmids, the introduction of such vectors and plasmids into host cells, and the expression of polypeptides from such vectors and plasmids in host cells. Such methods are well known to those of ordinary skill in the art.

### Example 1

Determining CHO Sequences that are Differentially Expressed Under Different Culture Temperatures

### Example 1.1

### Preparing Pools of Target Nucleic Acids

For each time point and temperature tested, duplicate cultures were seeded at 2×10⁵ cells/ml in appropriate serum-free chemically-derived media and either immediately cultured at 31° C. or allowed to grow for 24 hrs at 37° C. before culture at 31° C. (to increase cell mass). Cells were not split or fed. After 2 or 5 days, 1×10⁷ cells were harvested from each culture. Using well-known methods, total RNA was isolated from each population of CHO K1 cells cultured at 37° C. (control) for 2 or 5 days, and from each population of CHO K1 cells cultured at 31° C. for 2 or 5 days. The total RNA was converted to biotinylated cRNA for hybridization to the oligonucleotide array made according to U.S. patent application Ser. Nos. 11/128,049 and 11/128,061. Briefly, total RNA was isolated using the RNeasy Kit (Qiagen, Valencia, Calif.) according to the manufacturer's protocol. The isolated total RNA (5 μg) was then annealed to an oligo-dT primer (100 pMoles) in a reaction containing the BAC pool control reagent by incubation at 70° C. for 10 min. The primed RNA was subsequently reverse transcribed into complementary DNA (cDNA) by incubation with 200 units of Superscript RT II™ (Invitrogen, Carlsbad, Calif.) and 0.5 mM each dNTP (Invitrogen) in 1× first-strand buffer at 50° C. for 1 hr. Second-strand synthesis was performed by the addition of 40 units DNA Pol I, 10 units E. coli DNA ligase, 2 units RNase H, 30 μl second-strand buffer (Invitrogen), 3 μl of 10 mM dNTP (2.5 mM each) and dH₂0 to a 150 μl final volume, and incubation at 15° C. for 2 hrs. T4 DNA polymerase (10 units) was then added for an additional 5 min. The reaction was stopped by the addition of 10 μl of 500 mM EDTA. The resulting double-stranded cDNA was purified using a cDNA Sample Cleanup Module (Affymetrix). The cDNA (10 μl) was transcribed in vitro into cRNA by incubation with 1750 units of T7 RNA polymerase and biotinylated rNTPs at 37° C. for 16-20 hrs. Biotinylated rNTPs were used to incorporate biotin into the resulting cRNA. The biotinylated cRNA was then purified using the cRNA Sample Cleanup Module (Affymetrix) according to the manufacturer's protocol, and quantified using a spectrophotometer.

### Example 1.2

### Hybridization of Target Nucleic Acids to an Oligonucleotide Array

Biotin-labeled cRNA (15 μg) was fragmented for 35 min at 95° C. in 40 μl of 1× Fragmentation Buffer (Affymetrix). The fragmented cRNA was diluted in hybridization fluid [260 μl 1× MES buffer containing 300 ng herring sperm DNA, 300 ng BSA, 6.25 μl of a control oligonucleotide used to align the oligonucleotide array (e.g., Oligo B2, commercially available from Affymetrix, used to align Affymetrix arrays of oligonucleotide probes), and 2.5 μl standard curve reagent (as described in Hill et al. (2000) Science 290:809-12)] and denatured for 5 min at 95° C., followed immediately by incubation for 5 min at 45° C. Insoluble material was removed by a brief centrifugation, and the hybridization mix was added to the oligonucleotide array described in U.S. patent application Ser. Nos. 11/128,049 and 11/128,061. Target nucleic acids were allowed to hybridize to complementary oligonucleotide probes by incubation at 45° C. for 16 hrs under continuous rotation at 60 rpm. After incubation, the hybridization fluid was removed and the oligonucleotide array was extensively washed with 6×SSPET and 1×SSPET using protocols known in the art.

### Example 1.3

### Detection and Analysis of the Hybridization Profile Resulting from Hybridizing the Pool of Target Nucleic Acids to the Oligonucleotide Array

The raw fluorescent intensity value of each gene was measured at a resolution of 3 μm with an Agilent GeneArray Scanner. Microarray Suite (Affymetrix, Santa Clara, Calif.), which uses an algorithm to determine whether a gene is “present” or “absent,” as well as the specific hybridization intensity values of each gene on the array, was used to evaluate the fluorescence data. The expression value for each gene was normalized to frequency values by referral to the expression value of 11 control transcripts of known abundance that were spiked into each hybridization mix according to the procedure of Hill et al. (2001) Genome Biol. 2(12):research0055.1-0055.13 and Hill et al. (2000) Science 290:809-12, both of which are incorporated by reference herein in their entirety. The frequency of each gene was calculated and represents a value equal to the total number of individual gene transcripts per 10⁶ total transcripts.

Each condition and time point was represented by four biological replicates. Quadruplicate biological replicates were assayed for each time point. Each replicate was assayed in duplicate. Then the entire experiment was repeated. Programs known in the art, e.g., GeneExpress 2000 (Gene Logic, Gaithersburg, Md.), were used to analyze the presence or absence of a target sequence and to determine its relative expression level in one cohort of samples (e.g., condition or time point) compared to another sample cohort. A probeset called present in all replicate samples was considered for further analysis. Generally, fold-change values of two-fold or greater were considered statistically significant if the p values were less than or equal to 0.05.

Genes were identified using the expression profile program GeneExpress 2000. Unknown sequences were searched by blast homology search. Several genes were identified in which the transcriptional activity of the gene was increased when the culture temperature was lowered. The expression levels of eleven genes were altered more than two-fold (p value<0.05). Of the eleven genes, five demonstrated decreased levels of RNA expression at 37° C. over time but had a steady level of expression at 31° C. (“cold-induced genes”; data not shown). The remaining six genes demonstrated increased levels of RNA expression over time when CHO cells were cultured at 31° C. (data not shown). Two cold-induced genes, hamster mammary tumor-7 (HMT-7; also referred to as RIKEN 0610037N19 or N19) and layilin were selected for further analysis.

The HMT-7 coding region is 91% identical to MMT-7 and 89% identical to RMT-7. The accession numbers for RMT-7 and MMT-7 are AF465614 and NM_{—}026159, respectively (Wang et al. (2001) Oncogene 20:7710-21; Katayama et al. (2005) Science 309:1564-66; Moise et al. (2004) J. Biol. Chem. 279:50230-42). Layilin was cloned from CHO K1 cells and is 100% homologous to the sequence found in accession number AF093673 (Borowsky and Hynes (1998) J. Cell. Biol. 143:429-42).

Induction of HMT-7 and layilin at temperatures lower than physiological temperatures were verified using real-time polymerase chain reaction and Northern blot analysis, as described in Example2.

### Example 2

### Characterization of HMT-7 and Layilin Genomic Sequences

### Example 2.1

### Real-Time Polymerase Chain Reaction

A nonquantitative reverse transcription polymerase chain reaction (RT-PCR) was initially used to partially clone the cDNA of each gene. The cDNA were cloned into the vector pBluescript KS(−) (Stratagene, La Jolla, Calif.) and in vitro transcripts generated from the cloned cDNA fragments were subsequently quantified. Oligonucleotide and Taq-man probes, based on these cDNA sequences, were designed. The nucleotide sequences and SEQ ID NOs: of the reverse and forward primers and Taqman probes are listed in Table 2.

TABLE 2
Nucleotide sequences and SEQ ID NOs of the forward and
reverse primers and Taqman probes for HMT-7 and layilin

| | | |
|---|---|---|
| HMT-7 | | |
| Forward Primer | 5′-TTCCCAGACCGATCCACAAT-3′ | SEQ ID NO: 8 |
| Reverse Primer | 5′-GGCTCCTCCTGCCATTCC-3′ | SEQ ID NO: 9 |
| Taqman Probe | 5′-CTGTGCTGGTGCCCATGGCCT-3′ | SEQ ID NO: 10 |
| Layilin | | |
| Forward Primer | 5′-TGCGTGGTGATGTACCATCAG-3′ | SEQ ID NO: 11 |
| Reverse Primer | 5′-GTCATTCCACTGGAACATGTATGAG-3′ | SEQ ID NO: 12 |
| Taqman Probe | 5′-CGGCACCACCTGGCATCGG-3′ | SEQ ID NO: 13 |

Total RNA isolated from parallel cultures of CHO cells at 37° C., 34° C., 31° C., or 28° C. was subjected to real-time polymerase chain reaction using quantified in vitro transcripts as a standard curve.

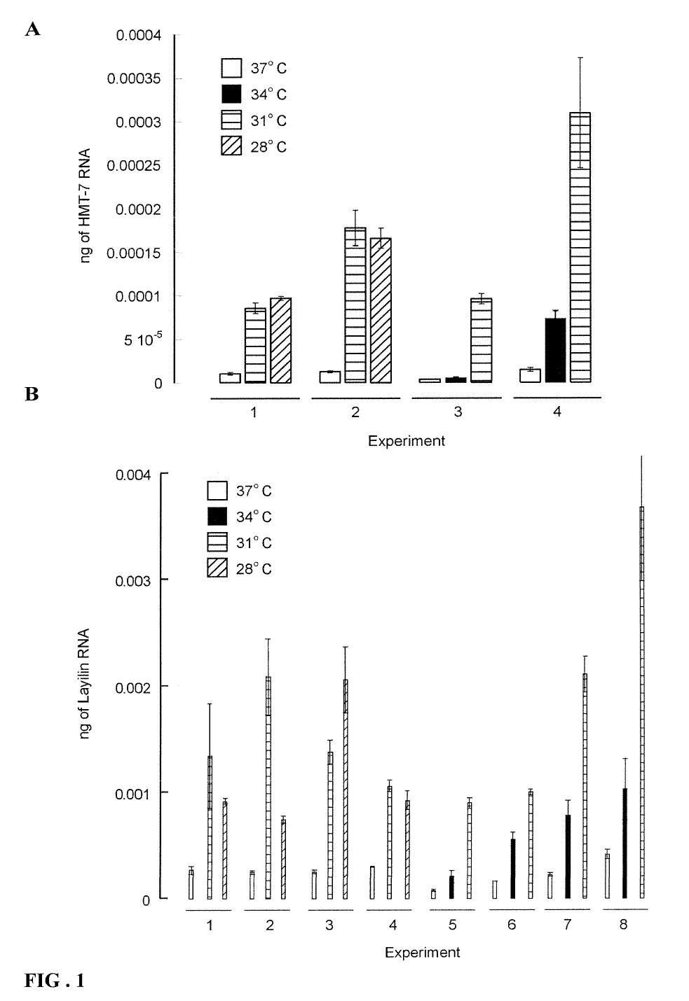

Shown in FIG. 1 are the amounts of either HMT-7 or layilin RNA transcribed by CHO cells grown at the different temperatures from four or eight different experiments. HMT-7 and layilin demonstrate eight-fold and four-fold increases, respectively, in expression in CHO cells grown at 31° C., compared to CHO cells grown at 37° C. (FIG. 1). Additionally, an increase in expression of either gene directly correlated with a decrease in temperature (FIG. 1).

### Example 2.2

### Northern Blot Analysis

RNA from cells cultured at 37° and 31° C. was subjected to Northern blot analysis using the isolated cDNA RT-PCR products as probes.

Briefly, RNA was isolated from seven individual CHO K1 cultures grown at 37° C. or 31° C. for 5 days. A total of 5 μg of RNA was separated and transferred to nylon membranes in the typical fashion (Ausubel et al. (1995) Current Protocols in Molecular Biology, Sects.). A cDNA fragment (50 ng) corresponding to the coding region of either HMT-7 or layilin was labeled with ³²P and used as a probe (described in further detail below). Hybridization of labeled polynucleotide to the membrane and subsequent washes of the membrane was done using Quickhyb Hybridization Solution (Stratagene, La Jolla Calif.) according to the manufacturer's instructions.

The probe used for HMT-7 (as set forth in SEQ ID NO:14) was a 334 by fragment of the RT-PCR product described in Example 2.1. It encodes for the exons between nucleotide 11514 and 12200 of the HMT-7 genomic sequence (SEQ ID NO:1) and spans three exons. The sequence overlaps that used in real-time PCR experiments. The layilin probe (set forth in SEQ ID NO:15) is a 397 by probe and corresponds to bases 325 to 721 of the layilin mRNA sequence.

The Northern blot analysis for HMT-7 demonstrated two bands (one at 1.4 kb and the other at 3.0 kb), suggesting the possibility of an alternative splice site for this gene (data not shown). The Northern blot analysis confirmed the real-time PCR data described above; both HMT-7 and layilin demonstrated increased expression by CHO cells when the cells were cultured at 31° C. (data not shown).

### Example 3

### Characterization of the HMT-7 Gene and the Layilin Gene

### Example 3.1

### 5′ Rapid Amplification of cDNA Ends

To isolate the 5′ end of the transcript, 5′ rapid amplification of cDNA ends (5′ RACE) was performed on RNA prepared from cells cultured at 31° C. Resultant PCR products were isolated, cloned, and sequenced. Two 5′ RACE product sequences were experimentally recovered for layilin, and one of these exactly matched previously published sequences, implying that layilin has two transcriptional start sites. Both of these start sites are present at 31° C., and there is no evidence to suggest that one site is preferred over the other at reduced temperatures. The 5′ end of the HMT-7 gene product had no known homology with any sequences present in public nucleotide databases.

### Example 3.2

### Genewalking and Determination of Promoter Sequences

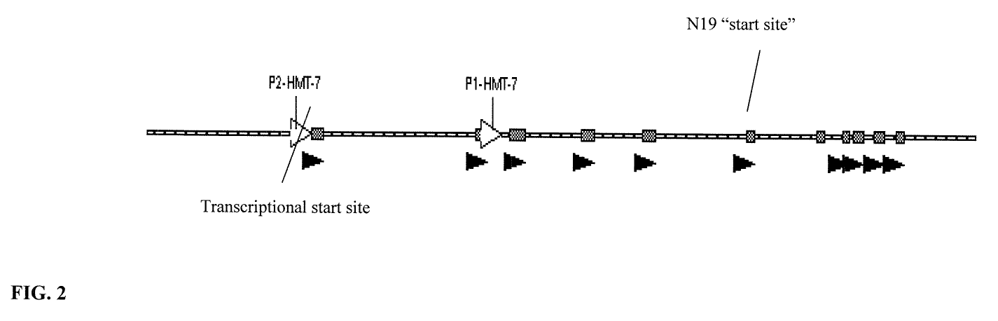

Both the layilin and HMT-7 5′ ends were then used as probes to screen a CHO specific genomic 8 phage library. No clones were isolated from the layilin screen, while two clones were successfully isolated and amplified from the HMT-7 screen. The genomic DNA fragment from the HMT-7 screen was >12 kb in size. A 3.5 kb genomic subfragment (i.e., portion) containing the previously isolated cDNA was cloned and sequenced. The remaining 5′ end of the HMT-7 gene was then isolated by genewalking as follows: CHO genomic DNA was isolated, digested to completion with four restriction enzymes, and then DNA linkers were ligated onto both the 5′ and 3′ ends of the resultant genomic DNA fragments. CHO-specific genomic DNA was then amplified by PCR using a gene-specific primer on the 3′ end and a linker-specific primer on the 5′ end. PCR products were isolated, subcloned into Topo-PCR II vector, and sequenced. This process was repeated until a ‘predicted promoter sequence’ (determined using algorithms employed by GRAIL software; Apocom Genomics, Knoxville, Tenn.) was identified. For HMT-7, two predicted promoter regions were identified (P1-HMT-7 and P2-HMT-7). In order to determine which is active at 31° C., primer extension experiments were performed, and the 5′ RACE experiments were repeated. As shown in FIG. 2, the active promoter sequence (P2-HMT-7) was found located at the most distal 5′ end of the HMT-7 gene. A sequence corresponding to a TATA box (a common feature of a promoter) was identified as located 33 bases 5′ to this transcriptional start site, further delineating P2-HMT-7 as the active promoter. However, the 5′ RACE results and Northern blot analysis demonstrating two transcripts, which are of appropriate size to indicate a full-length and a smaller putative splice variant and appear only in the temperature-induced samples, suggest that alternative splicing may be occurring. However, no sequence or transcript corresponding to the putative splice variant was isolated or cloned.

Genewalking was also used to isolate the genomic sequence for layilin, but as no clones were generated from the genomic library screen, the 5′ RACE product was used as the initial template. Resultant layilin PCR products were isolated, subcloned into Top-PCR II vector, and sequenced, and a predicted promoter sequence was identified.

The assembled full-length genomic sequence for HMT-7 is provided as SEQ ID NO:1. The assembled sequence includes genomic DNA isolated from the 5′ and 3′ untranslated regions (UTRs). The two predicted promoter regions are located at nucleotides 2422-2673 of SEQ ID NO:1 (i.e., SEQ ID NO:3) and 5615-5762 of SEQ ID NO:1 (i.e., SEQ ID NO:2). Additionally, Table 3 lists the positions of the exons within SEQ ID NO:1 for HMT-7.

TABLE 3
HMT-7 Exon Positions

| Exon | HMT-7 Exon Start | HMT-7 Exon Finish |
|---|---|---|
| 1 | 2664 | 2857 |
| 2 | 5346 | 5528 |
| 3 | 5895 | 6136 |
| 4 | 7052 | 7253 |
| 5 | 8054 | 8251 |
| 6 | 9740 | 9870 |
| 7 | 10880 | 11017 |
| 8 | 11297 | 11408 |
| 9 | 11487 | 11653 |
| 10 | 11814 | 11973 |
| 11 | 12166 | 12305 |

Set forth in SEQ ID NO:6 is the assembled 5′ genomic sequence for layilin, which includes the 5′ region 1341 bases upstream of the ATG coding for the start methionine (nucleotides 1341-1343). This 1341 base domain in layilin contains the predicted promoter sequence at nucleotides 223-1341. Similarly, the corresponding domain in the HMT-7 genomic sequence (i.e., nucleotides 1421-2685 of SEQ ID NO:1) contains the predicted promoter sequence(s) for HMT-7, e.g., for P2-HMT-7 at nucleotides 2422-2673.

### Example 4

### Creating a Temperature-inducible Expression Vector System Using the HMT-7 Promoter Region

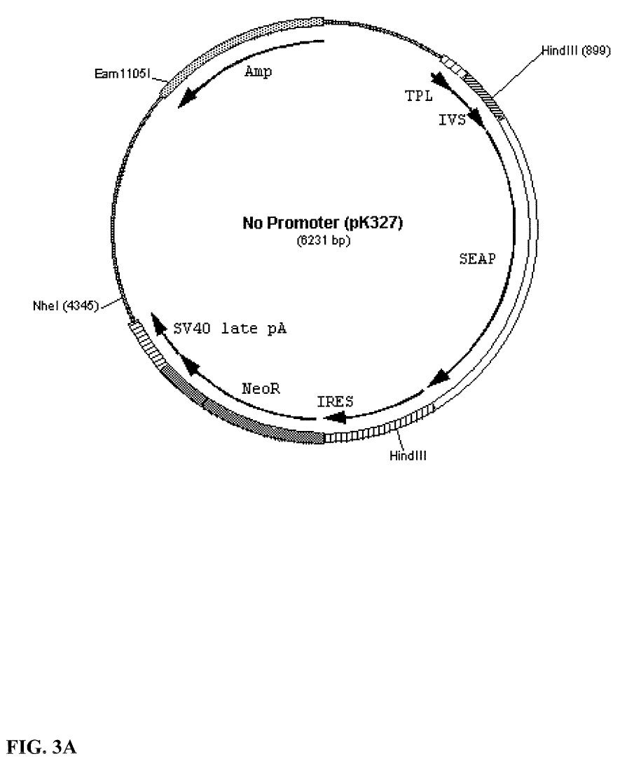

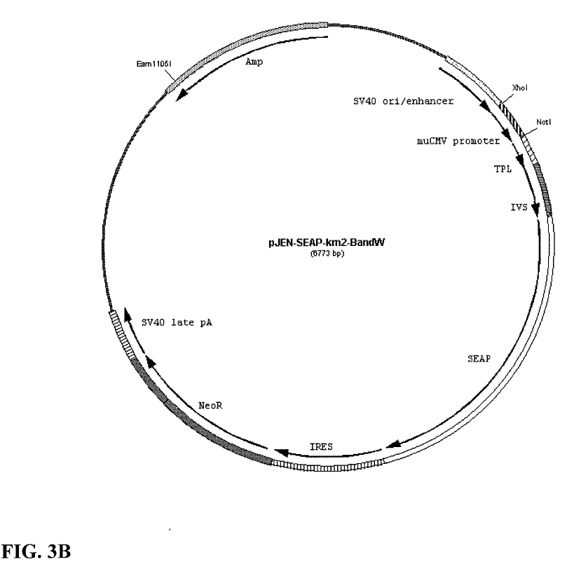

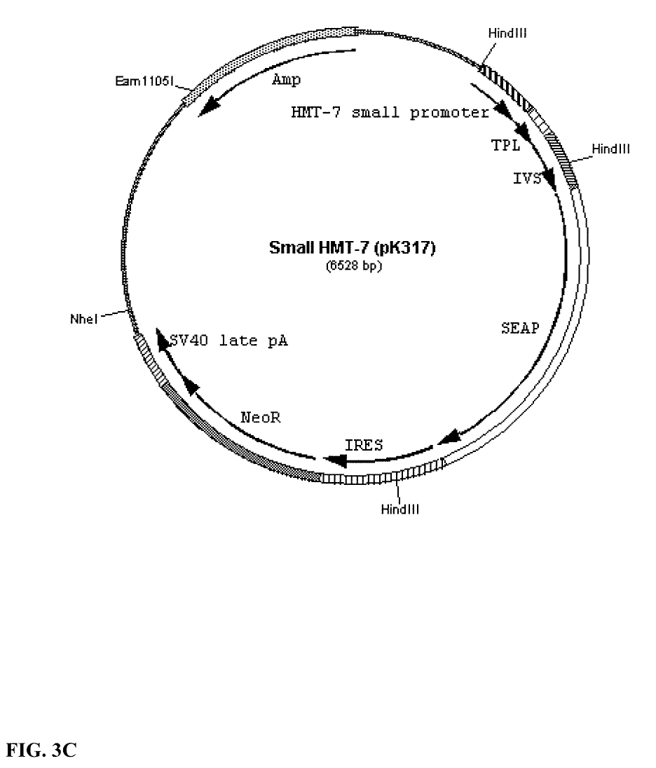

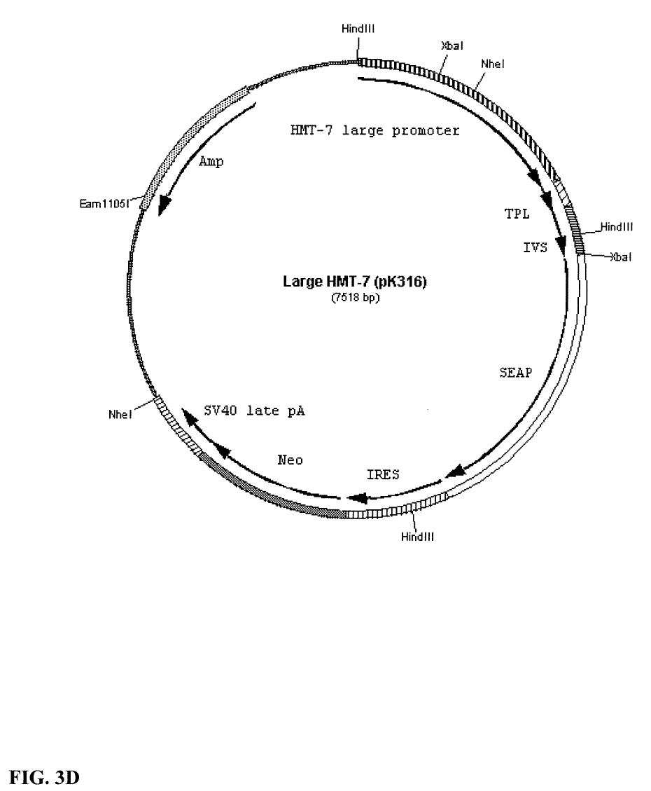

The promoter sequences characterized and described in Example 3 were isolated and placed upstream of the reporter gene human placental alkaline phosphatase (SEAP) (see FIGS. 3C and 3D). Briefly, either a small or large domain of the HMT-7 gene comprising P2-HMT-7 was placed upstream of SEAP. The sequence for the small domain corresponds to nucleotides 2404-2685 and the sequence for the large domain corresponds to nucleotides 1422-2685 of SEQ ID NO:1; these sequences are set forth in SEQ ID NO:16 and SEQ ID NO:17, respectively. A construct in which the SEAP reporter gene was not under the control of any promoter was generated to identify background expression caused by random integration. All constructs were linearized at the EAM1101 site and transfected into CHO K1 cells.

### Example 5

Testing the Inducible Expression Vectors Having and/or Consisting Essentially of the P2-HMT-7 Promoter

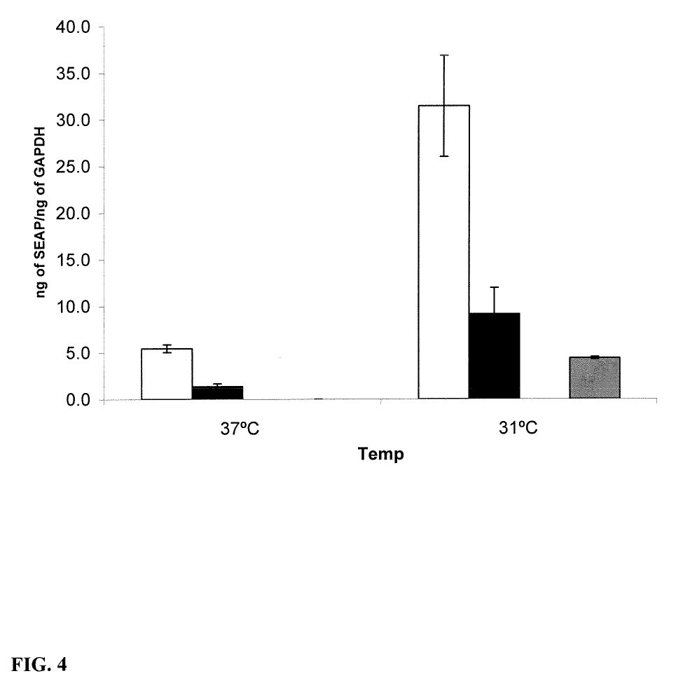

The expression vectors described in Example 4 were independently introduced into CHO K1 cells. Pools of transfected cells were allowed to grow to confluence under selection of G418 at 1 mg/ml. Duplicate sets of pools were seeded at 3×10⁵ cells/well and allowed to grow for seven days at either 37° C. or 31° C. After seven days, cells were harvested and RNA was isolated from all transfected cells. Triplicate samples of total RNA (100 μg) were assayed for SEAP expression or GAPDH expression using real-time PCR. FIG. 4 demonstrates that both a small and large domain of the HMT-7 gene comprising the P2-HMT-7 promoter have greater promoter activity at 31° C. than at 37° C.

In addition to testing pools of transfected cells, individual clones were selected using neomycin (G418), isolated and expanded. Each clone was seeded into a 96-well dish and allowed to grow for seven days at either 37° C. or 31° C. Clones were then washed, lysed and total RNA isolated using Qiagen RNEASY™ kit according to the manufacturer's instruction. SEAP and GAPDH RNA levels were obtained via real-time PCR with the oligos listed in Table 4.

TABLE 4
Oligos

| | | | SEQ |
|---|---|---|---|
| | | | ID |
| Target | Name | Sequence | NO: |
| GAPDH | mm0008 | 5′-TCCTTCCACAATGCCAAAGT-3′ | 18 |
| | mm0007 | 5′-CTGCACCACCAACTGCTTAG-3′ | 19 |
| | mmp0005 | 5′-CCCTGGCCAAGGTCATCCATG-3′ | 20 |
| SEAP | Seap 876 R | 5′-TTCCACACATACCGGGCAC-3′ | 21 |
| | Seap 813 F | 5′-TGGACGGGAAGAATCTGGTG-3′ | 22 |
| | Seap 837 T | 5′-AATGGCTGGCGAAGCGCCAG-3′ | 23 |

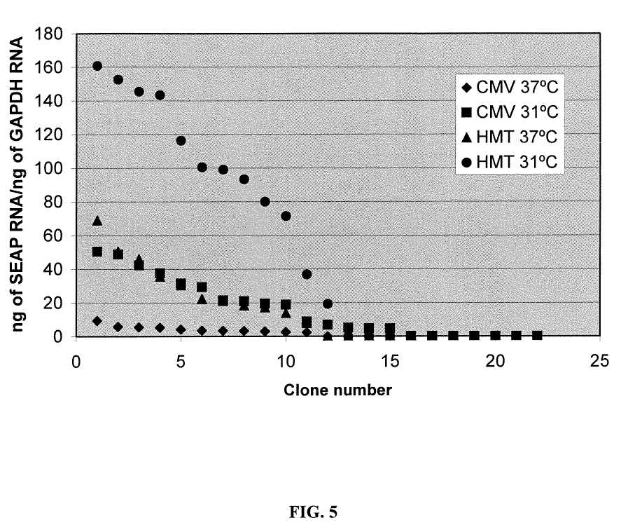

The quantity of GAPDH was also quantified to normalize for general fluctuations in RNA expression. GAPDH quantitation was also measured in triplicate samples using 100 ng of total RNA. Clones transfected with the HMT-7 reporter construct were slow to arise compared to clones transfected with the control promoter. Each clone was assayed at both 37° C. and 31° C. Shown in FIG. 5 is the production of SEAP RNA normalized to GAPDH expression by cells cultured at both temperatures for twenty-two clones transfected with alkaline phosphatase (SEAP) under the control of the CMV promoter and twenty clones transfected with alkaline phosphatase under the control of the large domain surrounding the P2-HMT-7 promoter. When grown at 31° C., clones transfected with SEAP under the control of the small domain comprising P2-HMT-7 demonstrated fold increases in alkaline phosphatase RNA that were comparable to clones transfected with SEAP under the control of the CMV promoter (data not shown). In contrast, the fold increases of alkaline phosphatase RNA production in clone number 1 through clone number 12 of cells transfected with alkaline phosphatase under the control of the large domain comprising P2-HMT-7 ranged from 1.7- to 9.6-fold when the cells were grown at 31° C. (FIG. 5). Similarly, although there was increased production of alkaline phosphatase by clones 13-16 of cells transfected with alkaline phosphatase under the control of the large domain comprising P2-HMT-7 at 31° C., the level of SEAP production by these clones was undetectable at 37° C., and thus, the fold change could not be calculated. Finally, clones 17-22 of cells transfected with alkaline phosphatase under the control of the large domain comprising P2-HMT-7 did not produce detectable levels of SEAP under either temperature. In contrast, the increase in alkaline phosphatase expression levels by cells transfected with SEAP under the control of the CMV promoter ranged from 1.8- to 9.1-fold. Consequently, it can be concluded that P2-HMT-7 and domains having and/or consisting essentially of the polynucleotide sequence of P2-HMT-7 (e.g., domains having the polynucleotide sequence set forth in SEQ ID NO:16 or SEQ ID NO:17) are temperature-induced promoters that may be used as part of an inducible mammalian expression system.

### Example 6

Testing the Inducible Expression Vectors Having and/or Consisting Essentially of the Layilin Promoter

The layilin promoter sequence characterized and described in Example 3 is isolated and placed upstream of the reporter gene human placental alkaline phosphatase (SEAP). The sequence for the predicted layilin promoter corresponds to nucleotides 223-1341 of SEQ ID NO:6, and is set forth in SEQ ID NO:7. Similar to Examples 4 and 5, above, a construct in which the SEAP reporter gene is not under the control of any promoter is generated to identify background expression caused by random integration. All constructs are linearized at the EAM1101 site and transfected into CHO K1 cells.

The expression vector containing the SEAP reporter gene under the control of the layilin promoter is introduced into CHO K1 cells. Pools of transfected cells grow to confluence under selection of G418 at 1 mg/ml. Duplicate sets of pools are seeded at 3×10⁵ cell/well and grow for seven days at either 37° C. or 31° C. After seven days, cells are harvested and RNA is isolated from all transfected cells. Triplicate samples of total RNA (100 μg) are assayed for SEAP expression or GAPDH expression using real-time PCR. In addition to pools of transfected cells, individual clones are selected using neomycin (G418), isolated and expanded. Each clone is seeded into a 96-well dish and grows for seven days at either 37° C. or 31° C. Clones are then washed, lysed and total RNA isolated using Qiagen RNEASY™ kit according to the manufacturers instruction. SEAP and GAPDH RNA levels are obtained via real-time PCR with the oligos listed in Table 4, above.

What is claimed is:

1. An isolated polypeptide comprising an amino acid sequence selected from the group consisting of the amino acid sequence of SEQ ID NO:5 and the amino acid sequence of an active fragment of SEQ ID NO:5.

2. An isolated nucleic acid molecule having a polynucleotide sequence that encodes the isolated polypeptide of claim 1.

3. The isolated nucleic acid molecule of claim 2, wherein the nucleic acid molecule has a polynucleotide sequence selected from the group consisting of the polynucleotide sequence of SEQ ID NO:4, the polynucleotide sequence of the complement of SEQ ID NO:4, the polynucleotide sequence of a subsequence of SEQ ID NO:4, and the polynucleotide sequence of the complement of a subsequence of SEQ ID NO:4.

4. The isolated nucleic acid molecule of claim 2, wherein the nucleic acid molecule is operably linked to at least one expression control sequence.

5. A host cell transformed or transfected with the nucleic acid molecule of claim 4.

6. A nonhuman transgenic animal in which the somatic and germ cells contain DNA having the isolated nucleic acid molecule of claim 4.

7. An isolated nucleic acid molecule that specifically hybridizes under highly stringent conditions to the isolated nucleic acid molecule of claim 2.

8. An antisense oligonucleotide complementary to an mRNA corresponding to the isolated nucleic acid molecule of claim 2.

9. An siRNA molecule comprising at least one strand of RNA, wherein the one strand has a polynucleotide sequence complementary to an mRNA corresponding to the isolated nucleic acid molecule of claim 2.

10. An isolated gene having the polynucleotide sequence of the isolated nucleic acid molecule of claim 2.

11. An isolated allele of the isolated gene of claim 10.

12. The isolated allele of claim 11 having a polynucleotide sequence selected from the group consisting of the polynucleotide sequence of SEQ ID NO:1, the polynucleotide sequence of the complement of SEQ ID NO:1, the polynucleotide sequence of a subsequence of SEQ ID NO:1, and the polynucleotide sequence of the complement of a subsequence of SEQ ID NO:1.

13. An isolated gene having a polynucleotide sequence that encodes hamster layilin.

14. An isolated allele of the isolated gene of claim 13.

15. The isolated allele of claim 14 having a polynucleotide sequence selected from the group consisting of the polynucleotide sequence of SEQ ID NO:6, the polynucleotide sequence of the complement of SEQ ID NO:6, the polynucleotide sequence of a subsequence of SEQ ID NO:6, and the polynucleotide sequence of the complement of a subsequence of SEQ ID NO:6.

16. An isolated temperature-induced promoter having a polynucleotide sequence selected from the group consisting of the polynucleotide sequence of an HMT-7 promoter and the polynucleotide sequence of a layilin promoter.

17. The isolated temperature-induced promoter of claim 16 having the polynucleotide sequence of SEQ ID NO:2, SEQ ID NO:3, SEQ ID NO:7, SEQ ID NO:16 or SEQ ID NO:17.

18. A host cell transformed or transfected with the isolated temperature-induced promoter of claim 16 or 17.

19. The host cell of claim 18, wherein the isolated temperature-induced promoter regulates the expression of a transgene.

20. The host cell of claim 18, wherein the host cell is a CHO cell.

21. An isolated polynucleotide comprising a nucleic acid sequence selected from the polynucleotide sequence of an HMT-7 promoter and the polynucleotide sequence of a layilin promoter.

22. The isolated polynucleotide of claim 21, wherein the nucleic acid sequence is SEQ ID NO:2 or SEQ ID NO:3.

23. A temperature-inducible mammalian expression vector comprising a temperature-induced promoter having a polynucleotide sequence selected from the group consisting of the polynucleotide sequence of an HMT-7 promoter and the polynucleotide sequence of a layilin promoter.

24. The temperature-inducible mammalian expression vector of claim 23, wherein the temperature-induced promoter has the polynucleotide sequence of SEQ ID NO:2, SEQ ID NO:3, SEQ ID NO:7, SEQ ID NO:16, or SEQ ID NO:17.

25. A host cell transformed or transfected with the temperature-inducible mammalian expression vector of claim 23 or 24.

26. The host cell of claim 25, wherein the host cell is a CHO cell.

27. A method of inducing transgene expression by a cell comprising the following steps:
(a) introducing an expression vector into the cell, wherein the expression vector comprises a mammalian temperature-induced promoter, and wherein the temperature-induced promoter regulates the expression of the transgene; and
(b) culturing the cell at an inducing temperature.

28. The method of inducing transgene expression by a cell of claim 27, wherein the temperature-induced promoter has the polynucleotide sequence of SEQ ID NO:2, SEQ ID NO:3, SEQ ID NO:7, SEQ ID NO:16 or SEQ ID NO:17.

29. The method of inducing transgene expression by a cell of claim 27 or 28, wherein the inducing temperature is below the physiological temperature of the cell.

30. The method of inducing transgene expression by a cell of claim 27 or 28, wherein the inducing temperature is in a range of 25° C. to 34° C.

31. A kit comprising a mammalian expression vector, wherein the mammalian expression vector comprises a temperature-induced promoter having a polynucleotide sequence selected from the group consisting of the polynucleotide sequence of an HMT-7 promoter and the polynucleotide sequence of a layilin promoter.

32. A hamster sequence differentially expressed under different culture conditions determined by a method comprising the steps of:
(a) forming a first hybridization profile and a second hybridization profile, wherein the first hybridization profile is formed by incubating target nucleic acids prepared from a first cell with a first hamster oligonucleotide array, wherein the second hybridization profile is formed by incubating target nucleic acids prepared from a second cell with a second hamster oligonucleotide array identical to the first hamster oligonucleotide array, and wherein the first cell differs from the second cell with respect to culture condition;
(b) detecting the first and the second hybridization profiles;
(c) comparing the first and second hybridization profiles; and
(d) determining at least one hamster sequence with a differential expression level in the first hybridization profile relative to its expression level in the second hybridization level.


SEQUENCE LISTING (23 sequences)

SEQ ID NO: 1 - DNA, 13466 bp, Hamster sp.
  misc_feature (59)..(60): n is a, c, g, or t
atcctaaccttgccaagccaaaatggtcaactctgtgtgtgagagagacagaaactctnngtgtgtgtgtgtgtgtgtgtgtgtgtgtgtgtgtgtgtgtgtgtgtnnagttgtttggggaatcaaatcctggttttcaacttatgtgtgcacacacacacatgcatgcatgcccccttgtgtgtgcgagaggattaagaggacaacctcagcaattgttacagcagagtctgttcagcttagtttttgagacaggatctctcatttgcctggaacccaacaagcaggcgggctggccagccaatgagctttagagatctgtctgtctccgctgtcctaagctgggattacaagtatgtgctaccacactaccacacccaattttcaaagaaattattatttttattcatgcagatgtatgtgtctgccatgtatgtacagatgtccatagggtactggatcccctgagctgaagttacagattgcttatcatatgtgagggcctgggttcaatctccaacactgaagggggaaaaaaggaaaaaaaaggaaagaaaaagatcagagataattctctgtatggtagtgtatgcccctggtcgcagaggcaggagaatcactgtaactctgacttatatagagttccaggccagctgaggctacatagcaagactttgtctcaataaaaaacacaataagggtaggtgaaatggctcagtaggtggaggtgcttcccagcaagtttgatgacctgggttcaatcccctgacggacccacatggtagaaggagagaaacaagttgtcttttgacctccatacatgcaccacgacggacaccattccactcacctccaactcaataagattttaaaaacaaaacaggggctggaaaaatggcttagtgggtaagagagcttgcttcaggtgtatggacctgagtttggctacctagcactatgggggatgaagataaaataattgctggttactggtcctagttccaggttcagttagagacccaacttgaaaggtataaggcacaaagtgacagaacagggcaactggcaatcctcctctggtgtttctgttcctatatgggcatgnncacacacacacacacacacacacacacacacacacnnacacacaaagagttgggtggaaaggctggtagcatagtagcaggacactagttcaccccttaaaactgaagggaagaaaatgtagatggagggttctgacgatcatacttcacaggcttagaggactaaggcagaaggcaggcctctccacagagtcagctctgacttggggaatataatctccattcctcaaggactggctgccagtaacaaaatgggatcattgtcgctttggggctgtgatctaggaaggatgtggtaatcctttacctgcagcactccagaactagctttctctccctggaacctcagcatccaactctctttttcctttcctggatgacagcacaggatcactcactcaaacacacacacacacacacacacacacacacacacacacacacaccagctaacctggcaggggtacaggtagatatgacagcggcagtctcgggaaggctttccggttactgtgtgggaaacaaaccaagtagcacagaatcatgacctgctttacaccgtcgactggagcactgctggggtctgtaaaaggcataaaatactcaaagttacaaagtcactcctcctgatagttggtatctgaagaaatgggcttcctataaaaacaatcggactcaaatcaagtgcttatttcagatctgttatcctcaaagggcaacgttaagaggataggctctctagactgtctgctggagctaacctccagaggtgatttagaagctggtttatccttaaatgcaaacacacacataggttagccttgacggtttttcttgagcttgttcctaggtggttctgtacttagaggccctgcagagtgattgcgattaggggatacagctagggacaacttcagggaccccgtgtttggctgctagccctggggcagtgacagactcaggagagcgcctgcgcagttctcaacaggggtctcaactcatccagaaacccagacacaggcggaggctgtgtgggaactcgggacggaaacgttctcattgcttgtaaagggaagtggaggaaagcaatgaattgcctccgaggtttcagcgaatccatcgcggaactgaacccagatcttctctgcctcccaggcggcatctctcttagaccagcaactttcacccttgtgtggctgcgcggtgaccatttccatcccacatcgtccccctcatcccccactccatctcaccccaccccaccccgggctcaccggacgcgtccccagcgttccggtaggagtcccgcatcccctgcggggcctcatcgcgcaggcgctcagcgcctcccgtcccgtgctagaactatatttcccagcaagcaatggaaatcccacacatcaaacgtcctacggagagccaggatggacaatgttcatcagctttgcgcgggcattggtcaaagtccctggcttttccttagtttggtgttcctctaatgacaaactagtggggcggagcttcagtcataaaagcggagctcagaagcaaaggatgcattcttcccaacccagactgcaagatgtggatctctgtgctactcctggcggtgctgctgctggcggtcctccgcagggtttacgtgggtctcttcggtggaagctccccgaaccccttcgcggaggatgtcaagcggccacctaaacccctggtgaccgacaaggaggctaggaagaaggttctcaaacaaggtgagctgcaggggctatgcgttccaggactgctctcgggccttctgctcatgcagggcacggaccccgtcttcccattagaaaagtgctggggaaactcacttgcatagggaggctccacatcttacagttctttgctcctgaaggtttgggtgcgggcactatccttggcacgccaagatgttggcataacctagccccagcccttctagttggaatctgttcagctaacagctcacacagaatgagaaacctttcccctacttgggtctagaaccaactttttctatgtccctgtccaacctcagggatatgggggagcagcacagaaaattttcccaacaagcggtttgacaggcaaagtgtaaccccagttcataggattgaagcttggttataaaacttgggagtttcactattgcttcaggataagagccctactatccagtcaaccagccatatttaaaacacgtgtagagttgcttccaaggtttatgggtgttttgtgtgtgtgtgtgtgtgtgtgtgtgtgtgtgtgtgttttattttatgtgcattggtgttttgcctgcatgtctgtctgaggatgccagatccccctggaacttgacttacagacagttgtaagatgccatgtgggtgctggggattgaaccgaggtcctctggaagagcagccagtgctcttaacccccgagccatctctccaacctccaaggtttttgtgtttttgaaacaggtctcacttttgcaccggagctggctccaaactcactataaagcccaggctcaaactttgagagtcctgcttttgcccacaggatgatggattagaagcatgtgccaacatgcctggctttcaaggttttaaagaattgttagagctgggcctagtggtgcaacattgtaattccagcctggactacaccatgaaacccagtctaaacaaacaccaaaaccctcaaatgtaaaaccttcaaaaaacaaacaaggatctcctgttggacttccaggggcctcttgcttgtaaagtttagcctaggctagtctcaactcaccatttattgggaactgaccttgaactactgatccttatgtgtctacttctaaggcttactacacccatttagtttattgttgttattttttgtggatttgaagagggaagaagatctcatacttgtagctcgggcaaatttagaacttgctatataggccaagtgtgcctcaaactcagtaattctcctgccttcagcaagagattacagatggtgggattacagataagagccaccatggcttaagcagtgattttaatattaattaattaattaatctatttatttagagacagtgtttctctatgttatcctgtatatagaccaggctggccttgaactcactgagattcttctgcctcctgagtgcttgggactaaagctgtgagcccctacacctggccatggacagtgatttttaatgaacaggttttgctggacatagtggcacatccctgtaatcccatgctggggaagaaaggcaggtggtctctacattgggagttccaggccagttagggctacacaatgaaaccctgtccccaataaattaaggaaaataaataaacaaagggggaaatgaagaaagtggacaggaggaaacaaccatggagtcttggaatgctttccagtttggttctgttaggtgtcctgccacaccagcgtcctccttttgtgaggctggtgctgtgacttctcagagtaatagctctgagcagccctgcctgccccacctctattgatattcatagaccttgcctcttctgaagaaacccagttccctgagccaccagcattccccttcccccattgttgtctttccttgcttgcattcccagaaacgactgcacttagattacaatacctgtactgttgccatctttcccgttttgttctaaagtagttaggtgattcctagtaggaaaggtatgaaaatccccaagtatggggggagggggcagggattgacatgtgaaacaagcttgtcctaatttgaataaaaaaaaattacaagaaaaaaagaacaaagaaaatccccaagtatatagtaggggttaagacccagttcatcactgtttaatctgcctgcctgagcctgtgtattccctgataaaatgttttgggacatcttagacaagagtcctgtgctaaattgccctagacagggatctgtctctctttctgtcctgttttgagactgggtctaaagagcccaggctggactccagtttagctgtgtaactgagcatgatcttgaactcctaatccaccttccaagtgcagggatcacagaattgcaccaccatgcccagcttgccttagagagttcttacatagtacaaactacagaagtcccctctggcttctctgactttgctgcctctttgctcttttctgagaagccaaccctctgtcttctcccttctacagcgttctcagtcagccgagtaccagggaacctggatgcagtggtgattggcagtggcattggggggctggcctcagctgcgattctggccaaagctggcaagagagtccttgtactggaacaacataccaaggcaggcggctgctgtcacacctttggggaaaatggccttgagtttgacactggtaagacttaagtgaaaaagggttgggagcatggctttgtctatggggggctaggaatgggcactttggggtgatatgagacacacacaataaagtaatatgagagtacctctatgtgttggtggtcccaacttctttttgggcatctccatttttgtctgacctggcaaatattgggaggtatatgttctggcatattcctatagaaagggtgggcagtcctctctgggcatgaacttggctaggcagggtaacctcttccttacttggcacatttccagggtgacaacctcttccttacttggcacatttccagggcccaattcccttatctttgtacctccttttctttccacttcccaacaggaatccattatattggacgcatgcaggagggcaacattggccgttttatcttggaccagatcactgaagggcaactggactgggcccccatggcctccccttttgacctgatgatactagaaggacccaatggccgaaaggagttccccatgtacagtgggagaaaacaatacatccagggccttaaggagaagttccccaaggaagaagctgtcattgacaagtacatggagctagttaaggtagcatgcaccatgatcagcatcctctatttccaacaagactgactttttcattggtgaagtaactggttaggagggactgagttggggaggcaggcaggaaggtccagtcatgtctgtcccgaatctagtgtacacttgtatatatgcctgtgccctgcctgactcttcagctgctaccctcacgctggcctctagatggtacccgtgggaagtcaggcttcgaggaagttagatgtcctctgagaggaaccagactctttgttgttttgtgatttttgagatagatgctcacaatgtagccctggctggcctggagctcacagagatcctcctgcctctgctggttgtatgccatcacacctggcttctggactgggatgtctgaaattgagggctggagatgaggctcatgtttagcatgtacaaaactctgaattagatcccagatgccccatagacagcatggtggtgtatcctgtaatcctagcactcaggaggcagaagtaggattaggagttcaaggtcactgggatatatgggactctatatagaaaaattattttgagcaggaaatggaagagcaaatatagtctcattggagtgtgaagaaagaaaaattcagggtccatttttttgttattgtttcgtggtgagggagataatcttgttatatagtcctggcttgggtgtcagtgtatagttccagttgcccttgaactcttggcagacctcctgccttagcatctcaactgctggcatcataaccctgagccaccatgcctgcccagcttaaatgtttttaagtggaaaaatccagacatgagtaagtgttgccaggtctgcagtcagtgccgactgttggtgaataggctacagcggttccagagactcactctacccttttttccccaggtggtagcccatggagcctctcatgccgtcctattgaagttcctcccgttgcccttgactcagctcctcaacaagttggggctgctgactcatttctctcctttctgccgagcatctactgagagcctggccgaggtcctgcagaagctaggggcttcccgtgagctccaggctgttctcagctacatcttccccacttacggtgggtggctgctggcccaggctcttctaagccactgccctcccagcttttggtctttcttcccatctgcctagctgacaagccagaatagaccccaggcctgtggataagggctgaagaagtctgacccccgctcgactctggtagaaactttctttttttaaatctactttctagatttctttttctacttctcccttttattttttttcttctctcccttttttttttccttcctttttggagaaaggctcttccaggcaagctttgaattcttgtagtcaaggatgtccctaaattcctaatccttctgcctccacctccccagtgctaggattacatgtccactaaggctggtggtgctggagatcaaatccaggcttccttgcatgttaggaaagcatcctgctaattgagctatatccccctccacatccctcacccctctaagacaggttctgactttgttcaggcttgtctggaactctctgtagcctagtctggctttgactcaatggtccttcttagtttttgagtgttgaggttgtaggtatgtactgtcacactgctcaagggccatttttctctagccaatagtattagcaggtcttggcaggatagtggttttcagggagtatatttatatcccactaagctctatatctgctctgggacttcaggagtatttggatcctgggatggactcatgtggccctttacaggaatgcctgtttcttcctgaatggtgccaagtgaccactcttgcctgctcatcatttctttccttcttcattctgtaggagtaacccctagctacaccaccttttccttgcatgctctgctggttgaccactacatacaaggggcattttatccacgaggaggttccagtgagattgccttccatatcatccctttgattcagcgggccgggggcgctgtcctcactagggccactgtacagagtgtgctactggactccactgggagagcctgtggtaagagacctgactgagctttggggtcagtgggctggggtgaaagggacctggggtctccctgtagatgaaccagcctatctgtgcaaggtaagaggctggtgagatggctcagtggataagtgttccagccactagacctgatgacctcagtttgattcccacagcctacatgttggaaggagagtactgaccctgcaagttgtcctctgacctccacatgtgggctgcagcaccctcaccaacacaaaatcaaggaagaaaaataccattgttacaaaaggtcaggtttgacataatggttttgtgagaatggccagagatgaggttggatacataggctacagcagaccttagaaactctggtgttttgtgaaggttgagtcatctggggagtattgtggacatagaacttggcagcaggagggtctgggactctatagaaccagtgttcttgggacggcaggactaaaagggacctaaaaaggtaagcaagtccatgtgggtgggtgagaagagctggagaagacaagcacagaagcatttgaggcaagggctgagcgactttgcagtgtacctggggcccgctttgattagaggaacttctaaggtctgaggaagacatctggatagtgtgtctatgacagacctaatcttttgaggcagggtctcaaattcactagctgaggcagtccttagaactcatgattgccccacttctacctcctaaatgctaagattacaggcatatgtctgtatcttgaagtatatcataccatgccatgactacaactaaagctttgagtgaaatgtatttcaagctttcctggatttatgttttatgggggataggaatgcactgaacctagggcctcacacatgctagacaagtgctataccactgagctatatcccctgcccttaagtgctgcttttccatcggcacagaggctggctgcactttatctgctgctgttttagccagcaaatgtggctccacagttactggccacttctctagcagtggccaggccactcaggctgtacctgtaggaatgctgttcctgcagacatgactgctgccaaatatagcttttaattaaatctagcttttaattaaatctattcttctacttatcaatgagactcaaaggctggggtgaaaacctgctagctcagagaggctgagtagcagctagctgaactgccaaaacaaaggacccatgctacactaagtccctccctactacttcctgtgcgtctatcatcttacagacaccctatgactctctatgattactttctgtcaactacttgctaactcagcctcctgacccaaggttgattttatttaattaatgcaaatgcaaactagggtttcacagtgtgatcaaatatcccacaacacttaagagagatctggggaaggttataggaccttttcttcctagagctcacagaagctcagccaaattcttgtgcttacaggtgtcagtgtgaagaagggacaagagctggtgaatatctattgccccgtggtcatctccaatgcaggaatgttcaatacctaccagcacttagtgccggagagtgcccgctgtctgccaggtaaaagggtggtcttctgaactgacatttcgtctgtggagcttataactcagcctggtgatgctccccctcttcctgggcacctccctggctgtctatctctggtctgtggcgccagcactgtaacgtttacatttgggaaacctaggaccacacaggtgaaaaatcccttctcagaggcaatggcagagctctgaggactctgtcatgtcagatgcaaatgtctaggctttattctcaggcctttgcctgagaaactgggactttaatagaaaaggactcacttaaagtgcagaagagggctggggatgaaacccagttggtgagagtgcttgcctggcatgcatcaagccctggatttgatccccagaaccagacaaggcagctgtggtggatttggctgtaatcccagcaccctagagggaggcaggaagataaattcaaagtcatccttggctatgagaccctgcctcaaaaaaacaaacaaaaaaaacaagggccactgaaatggctcaaagggtaaaaaagtatttgctgctaagtctggtaatgtgagttagatccccagagtagaagaagacaaccaagttgtcttctgtcttctccctgcttatccagacatgtacttgtccatgcacacccacaatttaaaaaaggaaagggaaaaaaatgaagaaaaatctaaacatcaaaggtgaggttgagacagcctgattatccagaggtgctgtgagttagctgtgctgggagccagcacctatgactaaggtacacagagcagaggtggtagaggtagaaaatgtctgaggctgtgtttgacatagccaggaagagacagaagagcaggtccctgccagggtcttgtagaaacagttgtaaaaatggctaggtctctatccaagagggcagctgagcagcaagctgcagattagaaaagggagcaccctgagactatcctgacagggagtagctgtctggtcacagctcctgcaacgcagtccccactggctttctctacagatgtgaagaagcagctggcaatggtacggcctggcatgagcatgctctcaatttcatttgcctgaaaggcaccaaggaggacgtgaagcttcagtccaccaactactatgtttattttgacacagacatggacaaagcgtaagatgcgagggggtggggcagactgatggcactgggccttaacctacaagaccctgcttccacctttgacatggttccaggaataggcagtaggatgtgtatagaggggggaggtgcacagtccccagtgtactcctaaaaaaccttttcatatcgaagctgtgtctgtttcacccctgtggggagttcctggaagtgaaccatcttttgggtggtactggtagcttggggcaagggggtggcctactgattggcagtcacaatttcctcatccacaggatgcaacactatgtctctatgcccaaggaaaaagctccagaacacattccccttcttttcattgcctctccatcaaccaaggacccaacctgggaggaccgattcccaggtagggctctaggtcctgggtagttgggtgtgggtcaaggcagggccaggcagcagtgacattgccttatacctgcagaccgatccacaatgactgtgctggtgcccatggccttcgagtggtttgaggaatggcaggaggagccaaagggcaagcggagtgctgactatgagaccctgaaacatgccttcatggaagcctccatgtccgtggtcatgaaactgttcccacagctggagggcaaggtaggggttgacactcgaaatgctggaggtcataacacacccctaactgcatttgtcttttcttgggttcagataaaggctgagataccaaagtcctaaggaaggattaggcccttcaaactcccttgctcacctttgctctgcccttctacctcctcaggtggagagtgtgactggaggatccccactgaccgaccagtactatctggctgctccccgaggtgctacctatggggctgaccatggcctggctcgtctgcatcctcgtgcagtggcttccctaagagcccaaacccccatccccaacctctacctgacaggtacactgcctcactttgccagaatctgggcttgcctgacagttatttgttcccatcctcaggccctgctgtccccttcagctccataccaggaactgggctgcctctttgggtggccttgatatggagggatgaacacaagaacccttgctctcccatcctcattcatgggccattgcttttgccttctaggccaagatacctttacctgtgggctgatgggggccctacaagtggccttgctgtgcagcagtgccatcctgaagcggaacttgtactcagatctgcaggctcttggctcaaaggttcagacacagaagaagaaggagtagtccattcagtgatatgctggaggaatggcactttctccaacttttctcatggtgtcctcctacagtgattccttgcacatataaccaaaaccactttgtttctactaactgctgtaaattgagtccttcacctagacctcttcccctttgtaccttgcatttctacttagggtctagtgtgggctacatagccttgatgactcaccatgaagaatgctttcattccttccccacacccaacactgagcaagggtcaggcacacagaaccccttagggtattggctattggattaggtaggtcaggtcctgccctgaagcactctgtactgtcaaagggaaggatgactagcgtgggtttggtgtggcaactctcaaagtgaggcaagaaactctcagccttaagttttgtcctctgatagaagcaggctggaggagcattccaatcaaagacatacaaaaagacaccactttatcaacaaaatgtcctatacaccagccagctgagatcttgactagagagagaatggtcagctaggtaaggaggagatgagagtggggacgttgggcctcaatacaggaagcccagagtgcaccaagcctagagatggcctttgtggttgttagcacatattaaaaactgagttttggtcgggcgtcgatggcgcacgcctttaatcccagcaatcaggaggaagaggcaggtgcatctctgtgagttcgaggccagcctggtctctagagcgagtgccaggataggctccaaagctacacagagagaccctgtctccaaaaaaaccaaataataataataataataataataataataataataataataataataataaaaacaaaaacaaacaaaaaaaacaaaccccaaaaaactgagttttgttttccaaaatgcttggaatcagaattccttcagattttatagcatttgcatacatataacttggggaacaagatccaggactaaacctcagattcgtttgtttcatatacatagaccagtcataatatcctaggtaatattttgaaaacttgtgagcattctatgacctgtcacatgagttcatgctattaaaaaaggcttgctttgggttttagaatagggattctcaacctatatgaataaattggcaagggggtaaaaa

SEQ ID NO: 2 - DNA, 147 bp, Hamster sp.
caataaagtaatatgagagtacctctatgtgttggtggtcccaacttctttttgggcatctccatttttgtctgacctggcaaatattgggaggtatatgttctggcatattcctatagaaagggtgggcagtcctctctgggcatg

SEQ ID NO: 3 - DNA, 251 bp, Hamster sp.
gcggggcctcatcgcgcaggcgctcagcgcctcccgtcccgtgctagaactatatttcccagcaagcaatggaaatcccacacatcaaacgtcctacggagagccaggatggacaatgttcatcagctttgcgcgggcattggtcaaagtccctggcttttccttagtttggtgttcctctaatgacaaactagtggggcggagcttcagtcataaaagcggagctcagaagcaaaggatgcattcttccc

SEQ ID NO: 4 - DNA, 1830 bp, Hamster sp.
  CDS (1)..(1827)
atgtggatctctgtgctactcctggcggtgctgctgctggcggtcctccgcagggtttacgtgggtctcttcggtggaagctccccgaaccccttcgcggaggatgtcaagcggccacctaaacccctggtgaccgacaaggaggctaggaagaaggttctcaaacaagcgttctcagtcagccgagtaccagggaacctggatgcagtggtgattggcagtggcattggggggctggcctcagctgcgattctggccaaagctggcaagagagtccttgtactggaacaacataccaaggcaggcggctgctgtcacacctttggggaaaatggccttgagtttgacactggaatccattatattggacgcatgcaggagggcaacattggccgttttatcttggaccagatcactgaagggcaactggactgggcccccatggcctccccttttgacctgatgatactagaaggacccaatggccgaaaggagttccccatgtacagtgggagaaaacaatacatccagggccttaaggagaagttccccaaggaagaagctgtcattgacaagtacatggagctagttaaggtggtagcccatggagcctctcatgccgtcctattgaagttcctcccgttgcccttgactcagctcctcaacaagttggggctgctgactcatttctctcctttctgccgagcatctactgagagcctggccgaggtcctgcagaagctaggggcttcccgtgagctccaggctgttctcagctacatcttccccacttacggagtaacccctagctacaccaccttttccttgcatgctctgctggttgaccactacatacaaggggcattttatccacgaggaggttccagtgagattgccttccatatcatccctttgattcagcgggccgggggcgctgtcctcactagggccactgtacagagtgtgctactggactccactgggagagcctgtggtgtcagtgtgaagaagggacaagagctggtgaatatctattgccccgtggtcatctccaatgcaggaatgttcaatacctaccagcacttagtgccggagagtgcccgctgtctgccaggtgtgaagaagcagctggcaatggtacggcctggcatgagcatgctctcaattttcatttgcctgaaaggcaccaaggaggacctgaagcttcagtccaccaactactatgtttattttgacacagacatggacaaagcgatgcaacactatgtctctatgcccaaggaaaaagctccagaacacattccccttcttttcattgcctctccatcaaccaaggacccaacctgggaggaccgattcccagaccgatccacaatgactgtgctggtgcccatggccttcgagtggtttgaggaatggcaggaggagccaaagggcaagcggagtgctgactatgagaccctgaaacatgccttcatggaagcctccatgtccgtggtcatgaaactgttcccacagctggagggcaaggtggagagtgtgactggaggatccccactgaccaaccagtactatctggctgctccccgaggtgctacctatggggctgaccatgacctggctcgtctgcatcctcgtgcagtggcttccctaagagcccaaacccccatccccaacctctacctgacaggccaagatacctttacctgtgggctgatgggggccctacaagtggccttgctgtgcagcagtgccatcctgaagcggaacttgtactcagatctgcaggctcttggctcaaaggttcagacacagaagaagaaggagtag

SEQ ID NO: 5 - protein, 609 aa, Hamster sp.
MWISVLLLAVLLLAVLRRVYVGLFGGSSPNPFAEDVKRPPKPLVTDKEARKKVLKQAFSVSRVPGNLDAVVIGSGIGGLASAAILAKAGKRVLVLEQHTKAGGCCHTFGENGLEFDTGIHYIGRMQEGNIGRFILDQITEGQLDWAPMASPFDLMILEGPNGRKEFPMYSGRKQYIQGLKEKFPKEEAVIDKYMELVKVVAHGASHAVLLKFLPLPLTQLLNKLGLLTHFSPFCRASTESLAEVLQKLGASRELQAVLSYIFPTYGVTPSYTTFSLHALLVDHYIQGAFYPRGGSSEIAFHIIPLIQRAGGAVLTRATVQSVLLDSTGRACGVSVKKGQELVNIYCPVVISNAGMFNTYQHLVPESARCLPGVKKQLAMVRPGMSMLSIFICLKGTKEDLKLQSTNYYVYFDTDMDKAMQHYVSMPKEKAPEHIPLLFIASPSTKDPTWEDRFPDRSTMTVLVPMAFEWFEEWQEEPKGKRSADYETLKHAFMEASMSVVMKLFPQLEGKVESVTGGSPLTNQYYLAAPRGATYGADHDLARLHPRAVASLRAQTPIPNLYLTGQDTFTCGLMGALQVALLCSSAILKRNLYSDLQALGSKVQTQKKKE

SEQ ID NO: 6 - DNA, 1395 bp, Hamster sp.
cgacggcccgggctggtaaaagattcattttcattttgtgtttgagtgtttttcttgcatttttttatgtgtgtgcaccacatgtgagcctggtgccccctttgggctggaagaggccaggagatcccttggaactcttgttagagccatcacactggtgctgggatctgaacccaggttttaggaaagagtaatgagtgctcttaaccactaagccatctctccagccccatctctgcttttctgattgctcctctgaaaagctagataaataagtaaagagaagcacactggaaccaaaatctagaagtccgtagctcagatgtggcttctgctatggttcacaacaccatagcctagaggaaaggcaaagaaaccttggctttttacttagttcatagtttgttgttgctgtcctttggtttttggtttttttgttttgtttttggacaggaaactggttctattgtgtgaatttgaaaacacattcactaaaagacacaaatataaacaactattgtgggagttttaaggggagcgtgcacttaggctacattggttatggaagctttaatggggggtaggatcattctttcctaggagagtcatctttctgtccaccaaaccactcccacctgccaatgtgtcctgcacaaagcagatggctttctggctgagatgtggaacactagcttctggtggaggcctcttaggaactcctgctctacctcattttgtggaagctcctcagtctgaagcactcctaggcagtagacacaccacctgcctgagaatccattcccctcatgttgtcttttcttgggtgcagcctgggtctgaagcttactgagatgtgagtaggggtcttgccactttggcccacttggtaagaactgggaatagtgataagggtgagaggttatactatttgaagatattgtaccctcttcctgggagttcagagactccctagtgggatttcactctctctcttaggcaaacccaggatctccctgggtggctctctccggagagctggccctgtcagctgccggctccctcctctaaacaagcactttctggagcctggagcgcagagcaccagatcaggccctcggggggcgggccagcaaaaggggctgccttccccgcagtgacgtcccaggaggcggaggggcaatcaactaacagactctgcgccctcccggactcctcccagtgcgggctctgtgtgtccttaagagatgcggctgcctcggtggcagttgcccgaacacagccgcctgtttgcccacttctctgccttagtcccggggtgttgggacgcactggagtccagcaatgcagccgggaccagcgttgcaggccgtgttgctggcggtgctgctgtcagaa

SEQ ID NO: 7 - DNA, 344 bp, Hamster sp.
ggcccacttggtaagaactgggaatagtgataagggtgagaggttatactatttgaagatattgtaccctcttcctgggagttcagagactccctagtgggatttcactctctctcttaggcaaacccaggatctccctgggtggctctctccggagagctggccctgtcagctgccggctccctcctctaaacaagcactttctggagcctggagcgcagagcaccagatcaggccctcggggggcgggccagcaaaaggggctgccttccccgcagtgacgtcccaggaggcggaggggcaatcaactaacagactctgcgccctcccggactcctcccagt

SEQ ID NO: 8 - DNA, 20 bp, Artificial
  HMT-7 Forward primer
ttcccagaccgatccacaat

SEQ ID NO: 9 - DNA, 18 bp, Artificial
  HMT-7 Reverse Primer
ggctcctcctgccattcc

SEQ ID NO: 10 - DNA, 21 bp, Artificial
  HMT-7 Taqman probe
ctgtgctggtgcccatggcct

SEQ ID NO: 11 - DNA, 21 bp, Artificial
  Layilin forward primer
tgcgtggtgatgtaccatcag

SEQ ID NO: 12 - DNA, 25 bp, Artificial
  Layilin reverse primer
gtcattccactggaacatgtatgag

SEQ ID NO: 13 - DNA, 19 bp, Artificial
  Layilin Taqman probe
cggcaccacctggcatcgg

SEQ ID NO: 14 - DNA, 334 bp, Artificial Sequence
  HMT-7 Northern blot probe
catggccttcgagtggtttgaggaatggcaggaggagccaaagggcaagcggagtgctgactatgagaccctgaaacatgccttcatggaagcctccatgtccgtggtcatgaaactgttcccacagctggagggcaaggtggagggtgtgactggaggatccccactgaccaaccagtactatctggctgctccccgaggtgctacctatggggctgaccatgacctggctcgtctgcatcctcgtgcagtggcttccctaagagcccaaacccccatccccaacctctacctgacaggccaagatatctttacctgtgggctgatgggggcc

SEQ ID NO: 15 - DNA, 397 bp, Artificial Sequence
  Layilin Northern blot probe
aacaacacagcctgccaggacctttatgcttggacagatgggagcacatcacaatttaggaactggtatgtggatgagccttcttgtggcagtgaggtctgcgtggtgatgtaccatcagccatcggcaccacctggcatcgggggctcatacatgttccagtggaatgacgaccggtgcaacatgaagaacaatttcatttgcaaatatgctgacgagaagccaagtacaacaccttctataaggcctggaggtgaagcaactgagccaccaacaccagtacttccagaagaaacacagaaagaagacaccaaagaaacattcaaagaaagcagagaggctgctttgaatcttgcctacatcctaatccccagcattcccctgttcctcttactag

SEQ ID NO: 16 - DNA, 282 bp, Hamster sp.
taggagtcccgcatcccctgcggggcctcatcgcgcaggcgctcagcgcctcccgtcccgtgctagaactatatttcccagcaagcaatggaaatcccacacatcaaacgtcctacggagagccaggatggacaatgttcatcagctttgcgcgggcattggtcaaagtccctggcttttccttagtttggtgttcctctaatgacaaactagtggggcggagcttcagtcataaaagcggagctcagaagcaaaggatgcattcttcccaacccagactgc

SEQ ID NO: 17 - DNA, 1264 bp, Hamster sp.
ctccagaactagctttctctccctggaacctcagcatccaactctctttttcctttcctggatgacagcacaggatcactcactcaaacacacacacacacacacacacacacacacacacacacacacaccagctaacctggcaggggtacaggtagatatgacagcggcagtctcgggaaggctttccggttactgtgtgggaaacaaaccaagtagcacagaatcatgacctgctttacaccgtcgactggagcactgctggggtctgtaaaaggcataaaatactcaaagttacaaagtcactcctcctgatagttggtatctgaagaaatgggcttcctataaaaacaatcggactcaaatcaagtgcttatttcagatctgttatcctcaaagggcaacgttaagaggataggctctctagactgtctgctggagctaacctccagaggtgatttagaagctggtttatccttaaatgcaaacacacacataggttagccttgacggtttttcttgagcttgttcctaggtggttctgtacttagaggccctgcagagtgattgcgattaggggatacagctagggacaacttcagggaccccgtgtttggctgctagccctggggcagtgacagactcaggagagcgcctgcgcagttctcaacaggggtctcaactcatccagaaacccagacacaggcggaggctgtgtgggaactcgggacggaaacgttctcattgcttgtaaagggaagtggaggaaagcaatgaattgcctccgaggtttcagcgaatccatcgcggaactgaacccagatcttctctgcctcccaggcggcatctctcttagaccagcaactttcacccttgtgtggctgcgcggtgaccatttccatcccacatcgtccccctcatcccccactccatctcaccccaccccaccccgggctcaccggacgcgtccccagcgttccggtaggagtcccgcatcccctgcggggcctcatcgcgcaggcgctcagcgcctcccgtcccgtgctagaactatatttcccagcaagcaatggaaatcccacacatcaaacgtcctacggagagccaggatggacaatgttcatcagctttgcgcgggcattggtcaaagtccctggcttttccttagtttggtgttcctctaatgacaaactagtggggcggagcttcagtcataaaagcggagctcagaagcaaaggatgcattcttcccaacccagactgc

SEQ ID NO: 18 - DNA, 20 bp, Artificial Sequence
  GAPDH mm0008 primer
tccttccacaatgccaaagt

SEQ ID NO: 19 - DNA, 20 bp, Artificial Sequence
  GAPDH mm0007 primer
ctgcaccaccaactgcttag

SEQ ID NO: 20 - DNA, 21 bp, Artificial Sequence
  GAPDH mmp0005 probe
ccctggccaaggtcatccatg

SEQ ID NO: 21 - DNA, 19 bp, Artificial Sequence
  SEAP 876 R primer
ttccacacataccgggcac

SEQ ID NO: 22 - DNA, 20 bp, Artificial Sequence
  SEAP 813 F primer
tggacgggaagaatctggtg

SEQ ID NO: 23 - DNA, 20 bp, Artificial Sequence
  SEAP 837T probe
aatggctggcgaagcgccag